(19) United States
(12) Patent Application Publication
(10) Pub. No.: US 2023/0282360 A1
(43) Pub. Date: Sep. 7, 2023

(54) SYSTEMS AND METHODS FOR USING MACHINE LEARNING TO PREDICT GENETIC MUTATION

(71) Applicant: ZS Associates, Inc., Evanston, IL (US)

(72) Inventors: Preeti Premraj, Bengaluru (IN); Shankar Viswanathan, Bengaluru (IN); Aaron Mitchell, Piedmont, CA (US); Pranava Goundan, San Francisco, CA (US); Tameesh Sood, New Delhi (IN); Shalvi Sheth, Pune (IN); Terence Wong, San Diego, CA (US); Dhanvi Shekhar, New Delhi (IN)

(73) Assignee: ZS Associates, Inc., Evanston, IL (US)

(21) Appl. No.: 18/115,944

(22) Filed: Mar. 1, 2023

(30) Foreign Application Priority Data
Mar. 2, 2022 (IN) 202241011357

(51) Int. Cl. G16H 50/30; G16H 30/20

(52) U.S. Cl. CPC G16H 50/30; G16H 30/20

(57) ABSTRACT

Disclosed herein are methods and systems for identfying genetic mutation and molecular alterations via imaging and clinical proxies using machine learning techniques. A processor can receive an image of a tumor of a patient. The processor can execute a first model to identify one or more visual attributes of the tumor using the image of the tumor as input. The processor can execute a second model to predict a genetic mutation or molecular alterations of the patient using the one or more visual attributes as input. The processor can identify a therapy protocol associated with the tumor based on the genetic mutation.

### CROSS-REFERENCE TO RELATED APPLICATIONS

This application claims the benefit of priority to Indian Provisional Application No. 202241011357, filed Mar. 2, 2022, the entirety of which is incorporated by reference herein.

### TECHNICAL FIELD

This application relates generally to systems and methods for training, calibrating, and executing artificial intelligence models.

### BACKGROUND

Developing and identifying the right therapy or medications for the right patients is a big hurdle during patient recruitment for clinical trials and during patient treatment by healthcare provider (HCP). Developing and identifying the right therapy medications for the right patients can be difficult because of both development barriers and adoption barriers. Development barriers can include complex disease pathways, the emergence of OMICS data, lack of patient subtyping leading to drug resistance, the complexity of clinical trials, among other barriers. Adoption barriers can include limited precision medicine programs, lack of clinical guidelines for molecular therapy, and many more.

### SUMMARY

Traditionally to identify the right patient for the right therapy during clinical trials or clinical settings, patients are subjected to various molecular testing to identify the actual cause of molecular alteration, as not all patients within an indication have the required molecular alteration. For example, the process may include acquiring the tissue of a patient for molecular testing. Acquiring such tissue can be invasive (many times requiring either surgery or a biopsy) and is associated with increased cost, as there is a need to test for multiple molecular alterations.

The computer models discussed herein (also referred to as or include disease algorithms) can leverage public datasets (e.g., rich in clinical, genomics, histopathological, radiomics, and digital pathology data) to develop or create algorithms to assist with identifying patients with specific genetic mutations or molecular alterations leveraging clinical and imaging proxies. These proxies can be generated or updated using artificial intelligence (AI)/machine learning (ML) methods or techniques as discussed herein. Using such methods can improve the probability of identifying or finding the right patients with genetic mutations or molecular alterations for specific therapy action or medication (e.g., therapy protocol), reduce the burden (e.g., physical or monetary burden) on the healthcare providers such as by minimizing the testing involved according to the predicted potential genetic mutations or molecular alterations associated with the patients, minimize resources involved with testing the patients for various types of genetic mutations or molecular alterations, and/or improve accuracy or efficiency (e.g., minimize time) in identifying the therapy protocol for treating patients. Upon identification of the right patient for the right molecular therapy, executing the disease algorithm can involve systems biology modeling to identify co-aberrant molecular alterations that could lead to resistance in a specific molecular therapy (e.g., use a complex disease pathway by leveraging a database such as a graph or nodal database). Using systems biology modeling can help exclude patients with resistance to specific therapy protocols despite being positive for the genetic mutation or molecular alteration. Systems biology modeling can also enable the system to recommend patients for one or a combination of therapies. Otherwise, systems biology modeling can predict patients who do not respond to either mono or combination therapy due to co-abberrant gene mutations and/or termed as non-responders.

The disease algorithm can correspond to or be included as part of one or more models configured to perform the functionalities discussed herein. For example, a first model (e.g., a machine learning model), used or executed by a device (e.g., network device, analytics server, or another computing device), can be configured to extract one or more visual attributes or features from at least one input image, such as an image of a tumor of a patient. The device can retrieve attributes (e.g., patient attributes) of the patient (e.g., the same patient) such as clinical attributes or other characteristics of the patient from memory (e.g., from a database). The device can execute a second model (e.g., a machine learning model, such as a clustering model, or a rules engine) that uses the visual attributes and/or patient attributes as input to identify one or more genetic mutations of the patient that may have caused the tumor. The device can retrieve genetic information (e.g., genes of the patient) regarding the patient from memory (e.g., a database). The device can input the genetic mutation and/or genetic information into a third model. The third model can be configured to store and/or traverse a nodal graph with different nodes and edges between nodes to create biological pathways. Upon execution, the third model can traverse the nodal graph based on the genetic mutations and/or genetic information of the patient. The third model can identify a therapy protocol that corresponds with the traversal (e.g., the last node of the traversal). The processor can identify the therapy protocol and an identifier (e.g., a name) of the patient to a list of patients that the processor has previously identified for the same therapy protocol. The model (or a different model) can be executed by the device to process the one or more visual attributes to predict or determine the genetic mutation associated with the tumor that the patient may have. In some cases, the model may use the clinical information additionally or alternatively to the visual attribute(s) to predict the genetic mutation(s).

The disease algorithm can help in identifying the right patient for the right molecular therapy by leveraging clinical and imaging proxies associated with the patients (e.g., retrieved/obtained from public datasets or generated using the models) and thus reduce the burden on the healthcare providers (HCPs) and/or clinical trial principal investigators to identify the right therap(ies) or medication(s). Further, within a specific molecular therapy for a patient subtype, the disease algorithm can help in excluding patients with resistance and identifying responders vs. non-responders, for instance, according to the disease pathways. This will improve the overall trial success and help in the adoption process of the targeted molecular therapies in the clinical setting.

An aspect of this disclosure can be directed to a method. The method can include receiving, by a processor, an image of a tumor of a patient. The method can include executing, by the processor, a first model to identify one or more visual attributes of the tumor using the image of the tumor as input. The method can include executing, by the processor, a second model to predict a genetic mutation of the patient using the one or more visual attributes as input. The method can include identifying, by the processor, a therapy protocol associated with the tumor based on the genetic mutation.

In some implementations, the first model can be a machine learning model trained based on historical data of a plurality of images of tumors, each of the plurality of images of tumors associated with a list of one or more visual attributes. In some implementations, the method can include to execute the second model, the method can include comparing, by the processor, the one or more visual attributes to one or more templates, each template corresponding to a different type of genetic mutation.

In some implementations, to execute the second model, the method can include retrieving, by the processor, patient attributes of the patient from a database. The method can include executing, by the processor, the second model using the one or more visual attributes and the patient attributes as input. In some implementations, to execute the second model, the method can include determining, by the processor, a confidence score for the genetic mutation based on the one or more visual attributes and the patient attributes. The method can include selecting, by the processor, the genetic mutation based on the confidence score.

In some implementations, the method can include adding, by the processor, an identification of the patient to a list of patients for the therapy protocol. In some implementations, the therapy protocol can be a first therapy protocol. The method can include executing, by the processor using one or more patient attributes of the patient as input, a third model to predict a molecular alteration for the patient. The method can include determining, by the processor, the first therapy protocol for the patient responsive to the molecular alteration corresponding to a second therapy protocol.

In some implementations, the molecular alteration can indicate a resistance to the first therapy protocol. In some implementations, the third model can be a nodal data structure.

An aspect of this disclosure can be directed to a system. The system can include a processor. The processor can be configured to receive an image of a tumor of a patient. The processor can be configured to execute a first model to identify one or more visual attributes of the tumor using the image of the tumor as input. The processor can be configured to execute a second model to predict a genetic mutation of the patient using the one or more visual attributes as input. The processor can be configured to identify a therapy protocol associated with the tumor based on the genetic mutation.

In some implementations, the first model can be a machine learning model trained based on historical data of a plurality of images of tumors, each of the plurality of images of tumors associated with a list of one or more visual attributes. In some implementations, to execute the second model, the processor can be configured to compare the one or more visual attributes to one or more templates, each template corresponding to a different type of genetic mutation.

In some implementations, to execute the second model, the processor can be configured to retrieve patient attributes of the patient from a database. The processor can be configured to execute the second model using the one or more visual attributes and the patient attributes as input. In some implementations, to execute the second model, the processor can be configured to determine a confidence score for the genetic mutation based on the one or more visual attributes and the patient attributes. The processor can be configured to select the genetic mutation based on the confidence score.

In some implementations, the processor can be further configured to add an identification of the patient to a list of patients for the therapy protocol. In some implementations, the therapy protocol can be a first therapy protocol, and the processor can be further configured to execute, using one or more patient attributes of the patient as input, a third model to predict a molecular alteration for the patient. The processor can be configured to determine the first therapy protocol for the patient responsive to the molecular alteration corresponding to a second therapy protocol. In some implementations, the molecular alteration can indicate a resistance to the first therapy protocol.

An aspect of this disclosure can be directed to a method. The method can include receiving, by a processor, clinical information of a patient. The method can include identifying, by the processor, one or more features from an image of a tumor of the patient. The method can include predicting, by the processor, using a model, a genetic mutation of the patient according to the one or more features. The method can include determining, by the processor, a therapy protocol according to the clinical information and the genetic mutation of the patient.

In some implementations, the genetic mutation can be a first genetic mutation, and to determine the therapy protocol, the method can include predicting, by the processor, using the model, a second genetic mutation of the patient according to the one or more features. The method can include determining, by the processor, the therapy protocol according to a combination of the first genetic mutation and the second genetic mutation and the clinical information.

In some implementations, to predict a genetic mutation and determine the therapy protocol, the method can include predicting, by the processor, using the model, more than two genetic mutations of the patient according to the one or more features. The method can include determining, by the processor, immunotherapy or adoptive cell therapy as the therapy protocol according to more than two genetic mutations associated with the tumor of the patient.

These and other aspects and implementations are discussed in detail below. The foregoing information and the following detailed description include illustrative examples of various aspects and implementations, and provide an overview or framework for understanding the nature and character of the claimed aspects and implementations. This Summary is not intended to identify key features or essential features, nor is it intended to limit the scope of the claims included herewith. The drawings provide illustration and a further understanding of the various aspects and implementations, and are incorporated in and constitute a part of this specification. Aspects can be combined and it will be readily appreciated that features described in the context of one aspect of the invention can be combined with other aspects. Aspects can be implemented in any convenient form. For example, by appropriate computer programs, which may be carried on appropriate carrier media (computer readable media), which may be tangible carrier media (e.g., disks) or intangible carrier media (e.g., communications signals). Aspects may also be implemented using suitable apparatus, which may take the form of programmable computers running computer programs arranged to implement the aspect. As used in the specification and in the claims, the singular form of ‘a’, ‘an’, and ‘the’ include plural referents unless the context clearly dictates otherwise.

### BRIEF DESCRIPTION OF THE DRAWING FIGURES

Objects, aspects, features, and advantages of embodiments disclosed herein will become more fully apparent from the following detailed description, the appended claims, and the accompanying drawing figures in which reference numerals identify similar or identical elements. Reference numerals that are introduced in the specification in association with a drawing figure may be repeated in one or more subsequent figures without additional description in the specification to provide context for other features, and not every element may be labeled in every figure. The drawing figures are not necessarily to scale, emphasis instead being placed upon illustrating embodiments, principles, and concepts. The drawings are not intended to limit the scope of the claims included herewith.

FIG. 1A is a block diagram of embodiments of a computing device, in accordance with one or more implementations;

FIG. 1B is a block diagram depicting a computing environment comprising client devices in communication with cloud service providers, in accordance with one or more implementations;

FIG. 2 is a block diagram of an example system in which performance prediction management services may manage and streamline access by clients to resource feeds (via one or more gateway services) and/or software-as-a-service (SaaS) applications, in accordance with one or more implementations;

FIG. 3 is an example computing environment for the AI-backed patient and procedure identification system, in accordance with one or more implementations;

FIG. 4A is a flow diagram of an example method for predicting genetic mutation and identifying therapy protocol, in accordance with one or more implementations;

FIG. 4B is a flow diagram of another example method for predicting genetic mutation and determining therapy protocol, in accordance with one or more implementations;

FIG. 5 is a flow diagram of an example detailed method for predicting genetic mutation and determining therapy protocol, in accordance with one or more implementations;

FIG. 6 illustrates an example NCCN guideline for molecular tests associated with respective medicines and therapies, in accordance with one or more implementations;

FIGS. 7-14 illustrate examples of some key oncogenic mutations of a particular disease, in accordance with one or more implementations;

FIG. 15 illustrates an example comparison of genetic mutations in medical images between different patients, in accordance with one or more implementations;

FIG. 16 illustrates an example process for image processing to extract notable features associated with certain genetic mutations mutation(s), in accordance with one or more implementations;

FIG. 17 illustrates an example of AI/ML generated segmented masks for validation of digital imaging and communications in medicine (DICOM) images, in accordance with one or more implementations, in accordance with one or more implementations;

FIG. 18 illustrates examples of clinical proxies and imaging proxies of a patient, in accordance with one or more implementations; and

FIG. 19 illustrates an example user interface for identifying patients in a clinical trial setting, in accordance with one or more implementations.

The features and advantages of the present solution will become more apparent from the detailed description set forth below when taken in conjunction with the drawings, in which like reference characters identify corresponding elements throughout. In the drawings, reference numbers generally indicate identical, functionally similar, and/or structurally similar elements.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

Section A describes a computing environment that may be useful for practicing embodiments described herein; and

Section B describes the training and executing of AI/ML models for the AI-backed patient and procedure identification system.

### Section A: Computing Environment

Prior to discussing the specifics of embodiments of the systems and methods of an appliance and/or client, it may be helpful to discuss the computing environments in which such embodiments may be deployed.

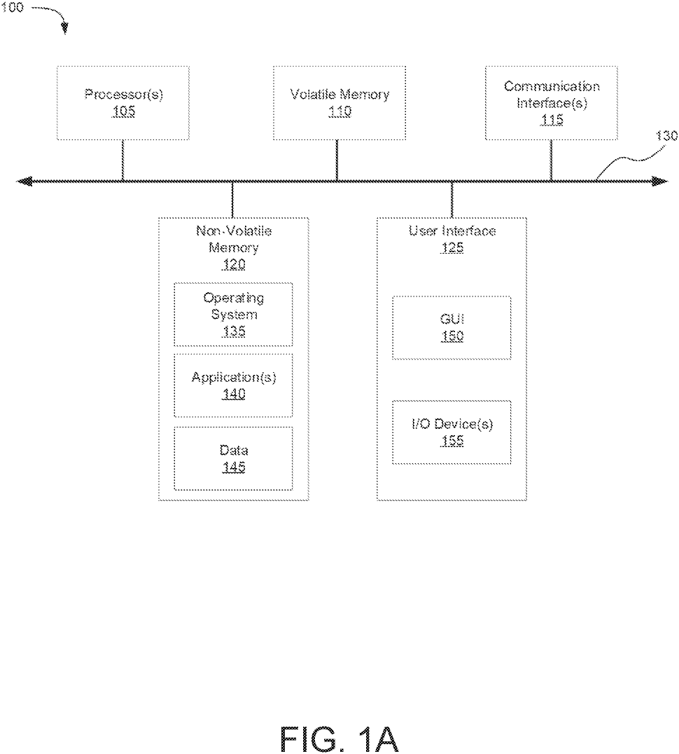

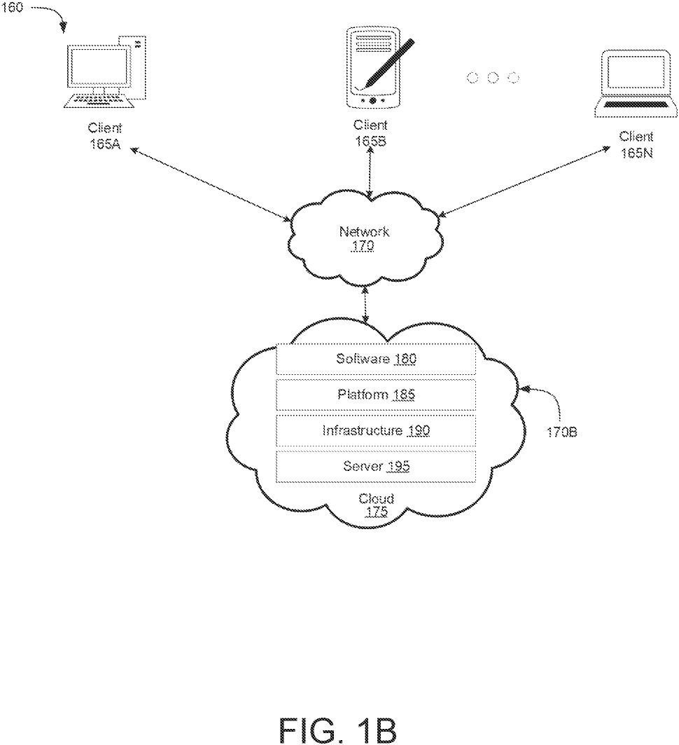

As shown in FIG. 1A, computer 100 may include one or more processors 105, volatile memory 110 (e.g., random access memory (RAM)), non-volatile memory 120 (e.g., one or more hard disk drives (HDDs) or other magnetic or optical storage media, one or more solid-state drives (SSDs) such as a flash drive or other solid-state storage media, one or more hybrid magnetic and solid-state drives, and/or one or more virtual storage volumes, such as cloud storage, or a combination of such physical storage volumes and virtual storage volumes or arrays thereof), user interface (UI) 125, one or more communications interfaces 115, and communication bus 130. User interface 125 may include a graphical user interface (GUI) 150 (e.g., a touchscreen, a display, etc.) and one or more input/output (I/O) devices 155 (e.g., a mouse, a keyboard, a microphone, one or more speakers, one or more cameras, one or more biometric scanners, one or more environmental sensors, one or more accelerometers, etc.). The non-volatile memory 120 stores operating system 135, one or more applications 140, and data 145 such that, for example, computer instructions of operating system 135 and/or applications 140 are executed by processor(s) 105 out of volatile memory 110. In some embodiments, volatile memory 110 may include one or more types of RAM and/or a cache memory that may offer a faster response time than the main memory. Data may be entered using an input device of GUI 150 or received from I/O device(s) 155. Various elements of computer 100 may communicate via one or more communication buses, shown as communication bus 130.

Computer 100 as shown in FIG. 1A is shown merely as an example, as clients, servers, intermediary, and other networking devices and may be implemented by any computing or processing environment and with any type of machine or set of machines that may have suitable hardware and/or software capable of operating as described herein. Processor(s) 105 may be implemented by one or more programmable processors to execute one or more executable instructions, such as a computer program, to perform the functions of the system. As used herein, the term “processor” describes circuitry that performs a function, an operation, or a sequence of operations. The function, operation, or sequence of operations may be hardcoded into the circuitry or soft coded by way of instructions held in a memory device and executed by the circuitry.

A “processor” may perform the function, operation, or sequence of operations using digital values and/or using analog signals. In some embodiments, the “processor” can be embodied in one or more application-specific integrated circuits (ASICs), microprocessors, digital signal processors (DSPs), graphics processing units (GPUs), microcontrollers, field-programmable gate arrays (FPGAs), programmable logic arrays (PLAs), multi-core processors, or general-purpose computers with associated memory. The “processor” may be analog, digital or mixed-signal. In some embodiments, the “processor” may be one or more physical processors or one or more “virtual” (e.g., remotely located or “cloud”) processors. A processor including multiple processor cores and/or multiple processors may provide functionality for parallel, simultaneous execution of instructions, or for parallel, simultaneous execution of one instruction on more than one piece of data.

Communications interfaces 115 may include one or more interfaces to enable computer 100 to access a computer network such as a Local Area Network (LAN), a Wide Area Network (WAN), a Personal Area Network (PAN), or the Internet through a variety of wired and/or wireless or cellular connections.

In described embodiments, the computing device 100 may execute an application on behalf of a user of a client computing device. For example, the computing device 100 may execute a virtual machine, which provides an execution session within which applications execute on behalf of a user or a client computing device, such as a hosted desktop session. The computing device 100 may also execute a terminal services session to provide a hosted desktop environment. The computing device 100 may provide access to a computing environment including one or more of one or more applications, one or more desktop applications, and one or more desktop sessions in which one or more applications may execute.

Referring to FIG. 1B, a computing environment 160 is depicted. Computing environment 160 may generally be implemented as a cloud computing environment, an on-premises (“on-prem”) computing environment, or a hybrid computing environment including one or more on-prem computing environments and one or more cloud computing environments. When implemented as a cloud computing environment, also referred to as a cloud environment, cloud computing, or cloud network, computing environment 160 can provide the delivery of shared services (e.g., computer services) and shared resources (e.g., computer resources) to multiple users. For example, the computing environment 160 can include an environment or system for providing or delivering access to a plurality of shared services and resources to a plurality of users through the internet. The shared resources and services can include but are not limited to, networks, network bandwidth, servers, processing, memory, storage, applications, virtual machines, databases, software, hardware, analytics, and intelligence.

In some embodiments, the computing environment 160 may provide client 165 with one or more resources provided by a network environment. The computing environment 160 may include one or more clients 165a-165n, in communication with a cloud 175 over one or more networks 170. Clients 165 may include, e.g., thick clients, thin clients, and zero clients. The cloud 108 may include back-end platforms, e.g., servers, storage, server farms, or data centers. The clients 165 can be the same as or substantially similar to computer 100 of FIG. 1A.

The users or clients 165 can correspond to a single organization or multiple organizations. For example, the computing environment 160 can include a private cloud serving a single organization (e.g., enterprise cloud). The computing environment 160 can include a community cloud or public cloud serving multiple organizations. In some embodiments, the computing environment 160 can include a hybrid cloud that is a combination of a public cloud and a private cloud. For example, the cloud 175 may be public, private, or hybrid. Public clouds 108 may include public servers that are maintained by third parties to the clients 165 or the owners of the clients 165. The servers may be located off-site in remote geographical locations as disclosed above or otherwise. Public clouds 175 may be connected to the servers over a public network 170. Private clouds 175 may include private servers that are physically maintained by clients 165 or owners of clients 165. Private clouds 175 may be connected to the servers over a private network 170. Hybrid clouds 175 may include both the private and public networks 170 and servers.

The cloud 175 may include back-end platforms, e.g., servers, storage, server farms, or data centers. For example, the cloud 175 can include or correspond to a server or system remote from one or more clients 165 to provide third-party control over a pool of shared services and resources. The computing environment 160 can provide resource pooling to serve multiple users via clients 165 through a multi-tenant environment or multi-tenant model with different physical and virtual resources dynamically assigned and reassigned responsive to different demands within the respective environment. The multi-tenant environment can include a system or architecture that can provide a single instance of the software, an application, or a software application to serve multiple users. In some embodiments, the computing environment 160 can provide on-demand self-service to unilaterally provision computing capabilities (e.g., server time, network storage) across a network for multiple clients 165. The computing environment 160 can provide elasticity to dynamically scale out or scale in responsive to different demands from one or more clients 165. In some embodiments, the computing environment 160 can include or provide monitoring services to monitor, control, and/or generate reports corresponding to the provided shared services and resources.

In some embodiments, the computing environment 160 can include and provide different types of cloud computing services. For example, the computing environment 160 can include Infrastructure as a service (IaaS). The computing environment 160 can include Platform as a service (PaaS). The computing environment 160 can include server-less computing. The computing environment 160 can include Software as a service (SaaS). For example, the cloud 175 may also include a cloud-based delivery, e.g., Software as a Service (SaaS) 180, Platform as a Service (PaaS) 185, and Infrastructure as a Service (IaaS) 190. IaaS may refer to a user renting the use of infrastructure resources that are needed during a specified time period. IaaS providers may offer storage, networking, servers, or virtualization resources from large pools, allowing the users to quickly scale up by accessing more resources as needed. Examples of IaaS include AMAZON WEB SERVICES provided by Amazon.com, Inc., of Seattle, Washington; RACKSPACE CLOUD provided by Rackspace US, Inc., of San Antonio, Texas; Google Compute Engine provided by Google Inc. of Mountain View, California; or RIGHTSCALE provided by RightScale, Inc., of Santa Barbara, California. PaaS providers may offer functionality provided by IaaS, including, e.g., storage, networking, servers, or virtualization, as well as additional resources such as, e.g., the operating system, middleware, or runtime resources. Examples of PaaS include WINDOWS AZURE provided by Microsoft Corporation of Redmond, Washington; Google App Engine provided by Google Inc.; and HEROKU provided by Heroku, Inc., of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc.; SALESFORCE provided by Salesforce.com Inc. of San Francisco, California; or OFFICE 365 provided by Microsoft Corporation. Examples of SaaS may also include data storage providers, e.g., DROPBOX provided by Dropbox, Inc., of San Francisco, California; Microsoft SKYDRIVE provided by Microsoft Corporation; Google Drive provided by Google Inc.; or Apple ICLOUD provided by Apple Inc. of Cupertino, California.

Clients 165 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP and may use Representational State Transfer (REST) protocol or Simple Object Access Protocol (SOAP). Clients 165 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 165 may access SaaS resources through the use of web-based user interfaces, provided by a web browser (e.g., GOOGLE CHROME, Microsoft INTERNET EXPLORER, or Mozilla Firefox provided by Mozilla Foundation of Mountain View, California). Clients 165 may also access SaaS resources through smartphone or tablet applications, including, e.g., Salesforce Sales Cloud or Google Drive app. Clients 165 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as e.g., Advanced Encryption Standard (AES). Data resources may be sent over Transport Layer Security (TLS) or Secure Sockets Layer (SSL).

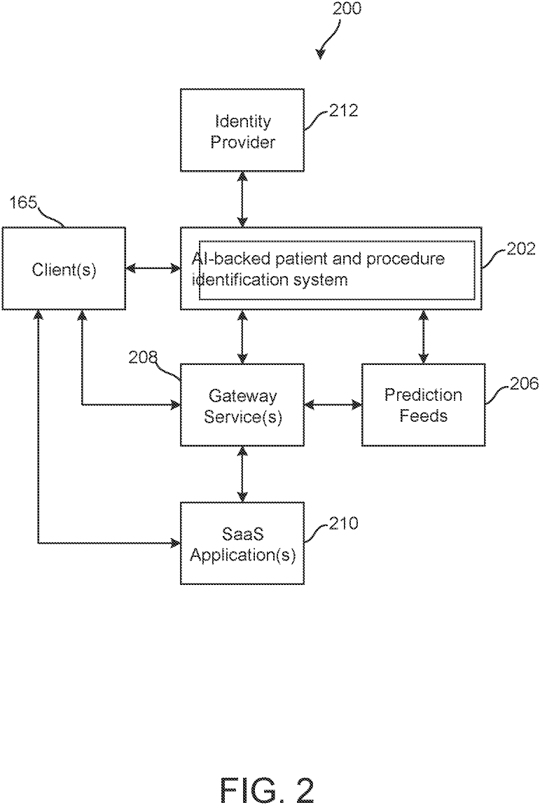

FIG. 2 is a block diagram of an example system 200 in which an AI-backed patient and procedure identification system 202 may manage and streamline access by one or more clients 165 to one or more prediction feeds 206 (via one or more gateway services 208) and/or one or more software-as-a-service (SaaS) applications 210. As used herein, a prediction feed is a result of the execution of one or more AI models discussed herein. In particular, the AI-backed patient and procedure identification system 202 may employ an identity provider 212 to authenticate the identity of a user of a client 165 and, following authentication, identify one or more prediction feeds the user is authorized to access. For the prediction feed(s) 206, the client 165 may input attributes associated with a product and may request access to one or more AI models via a gateway service 208. For the SaaS application(s) 210, the client 165 may access the selected application directly. The SaaS application(s) 210 may allow the client 165 to access the platform discussed herein and view the prediction feeds 206.

The client(s) 165 may be any type of computing device capable of accessing the prediction feed(s) 206 and/or the SaaS application(s) 210, and may, for example, include a variety of desktop or laptop computers, smartphones, tablets, etc. Each of the AI-backed patient and procedure identification system 202, the prediction feed(s) 206, the gateway service(s) 208, the SaaS application(s) 210, and the identity provider 212 may be located within an on-premises data center of an organization for which the system 200 is deployed, within one or more cloud computing environments, or elsewhere.

### Section B: Training and Executing the AI-Backed Patient and Procedure Identification System

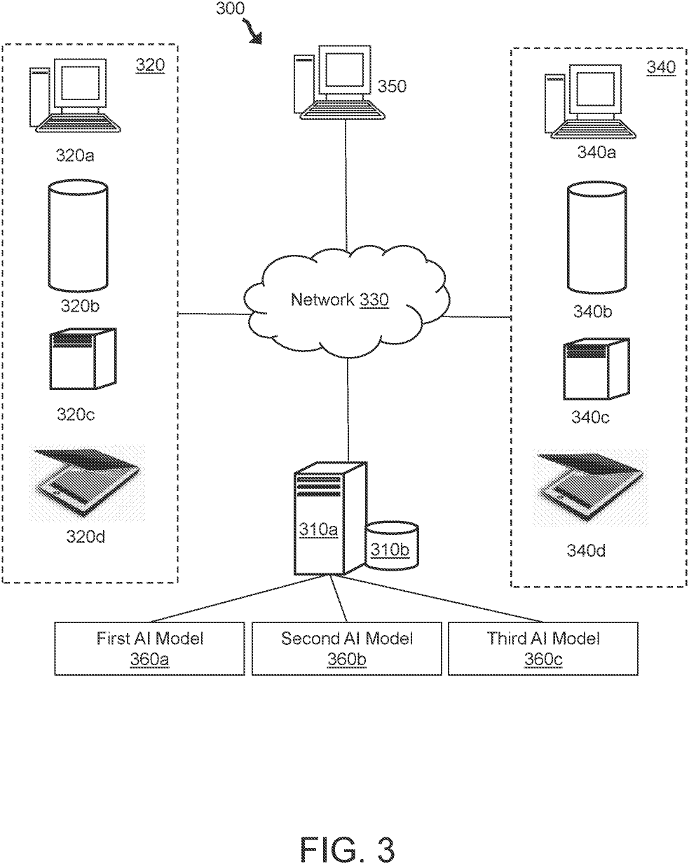

As will be described throughout, a server of an AI-backed patient and procedure identification system 300 (such as an analytics server 310a) can retrieve and analyze data using various methods described herein to predict which patient is a good candidate for a therapy. FIG. 3 is a non-limiting example of components of the system 300 in which the analytics server 310a operates. The analytics server may be any computer, server, or processor described in FIG. 1A-2.

The analytics server 310a may utilize features described in FIG. 3 to retrieve data and to generate/display results. The analytics server 310a is communicatively coupled to a system database 310b, electronic data sources 320a-d (collectively electronic data sources 320), end-user devices 340a-d (collectively end-user device 340), and an administrator computing device 350. The system 300 is not confined to the components described herein and may include additional or alternative components, not shown for brevity, which is to be considered within the scope of the embodiments described herein.

The above-mentioned components may be connected through a network 330. The examples of the network 330 may include but are not limited to, private or public LAN, WLAN, MAN, WAN, and the Internet. The network 330 may include both wired and wireless communications according to one or more standards and/or via one or more transport mediums.

The analytics server 310a may utilize one or more application programming interfaces (APIs) to communicate with one or more of the electronic devices described herein. For instance, the analytics server may utilize APIs to automatically receive data from the electronic data sources 320. The analytics server 310a can receive data as it is generated, monitored, and/or processed by the electronic data source 320. For instance, the analytics server 110a may utilize an API to receive click stream data from the database 320b without any human intervention. This automatic communication allows for faster retrieval and processing of data. In various implementations, the electronic data source 320 may include or correspond to public data sources. In such cases, the analytics server 310a can obtain, retrieve, or acquire other types of data from the electronic data source 320, such as public datasets for generating, training, or updating the model. In various cases, the analytics server 310a can obtain clinical information, among other data/information associated with patients for diagnostic or analysis purposes discussed herein.

The analytics server 310a may generate and/or host an electronic platform (AI-driven customer decision journey platform) having a series of graphical user interfaces (GUIs) configured to use various computer models (including artificial intelligence (AI) models) to project and display patient analysis data. The platform can be displayed on the electronic data sources 320, the administrator computing device 350, and/or end-user devices 340. An example of the platform generated and/or hosted by the analytics server 310a may be a web-based application or a website configured to be displayed on different electronic devices, such as mobile devices, tablets, personal computers, and the like. Even though certain embodiments discuss the analytics server 310a displaying the results, it is expressly understood that the analytics server 310a may either directly generate and display the platform described herein or may present the data to be presented on a GUI displayed on the end-user devices 340.

The analytics server 310a may host a website (also referred to herein as the platform) accessible to end-users operating any of the electronic devices described herein (e.g., end-users), where the content presented via the various webpages may be controlled based upon each particular user’s role or viewing permissions. The analytics server 310a may be any computing device comprising a processor and non-transitory machine-readable storage capable of executing the various tasks and processes described herein. Non-limiting examples of such computing devices may include servers, computers, workstation computers, personal computers, and the like. While this example of the system 300 includes a single analytics server 310a, in some configurations, the analytics server 310a may include any number of computing devices operating in a distributed computing environment.

The analytics server 310a may execute one or more software applications configured to display the platform (e.g., host a website), which may generate and serve various webpages to each electronic data sources 320 and/or end-user devices 340. Different end-users may use the website to view and/or interact with the predicted results.

The analytics server 310a may be configured to require user authentication based upon a set of user authorization credentials (e.g., username, password, biometrics, cryptographic certificate, and the like). In such implementations, the analytics server 310a may access the system database 310b configured to store user credentials, which the analytics server 310a may be configured to reference to determine whether a set of entered credentials (purportedly authenticating the user) match an appropriate set of credentials that identify and authenticate the user.

The analytics server 310a may also store data associated with each user operating one or more electronic data sources 320 and/or end-user devices 340. The analytics server 310a may use the data to determine whether a user device is authorized to view results generated by the AI models (e.g., first model 360a, second model 360b, or the third model 360c).

The analytics server 310a may receive patient data and perform various analysis using the first model 360a, the second model 360b, and/or the third model 360c. The AI models 360a-c are shown as separate models to illustrate that the methods and systems described herein are not limited to a single model. In some embodiments, the analytics server 310a may utilize one model that combines the functionality of the first to third models 360a-c. In some other embodiments, the analytics server 310a may divide some of the functionality discussed herein between the two depicted models. In yet some other embodiments, the analytics server 310a may divide some of the functionality discussed herein between more than two models. For example, the features or functionalities discussed herein can be implemented or executed via one or more models, including, but not limited to the AI models 360a-c.

In some implementations discussed herein, any one of the devices of the network 330 with access to at least the electronic data source 320 or clinical information of patients can be configured to generate, train, execute, or update the one or more AI models, such as the analytics server 310a, end-user devices 340, or administrator computing device 350. In some cases, different devices within the network 330 can include or execute different AI models, where a first network device can delegate certain functionalities of an AI model to at least one other network device for execution by another AI model.

In various configurations discussed herein, the systems and methods of the technical solution can train one or more AI models (e.g., via one of the devices within the network 330) using historical data from various patients as inputs (e.g., imaging data or clinical data) to identify the (notable) features of diseases (e.g., tumors) associated with the patients. The systems and methods can execute the one or more models using medical images or clinical information of the patients to generate an output, such as identifications (e.g., text or strings) that identify or describe genetic mutations, molecular alterations, or identified therapy protocols associated with/for the patients. In some cases, the output or results from the AI model(s) can be shared or provided to the HCP, patients, or others related to the input data/information.

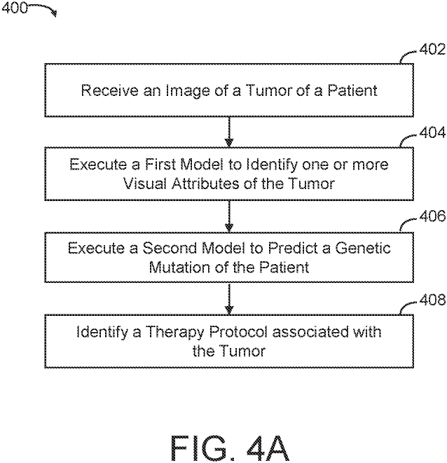

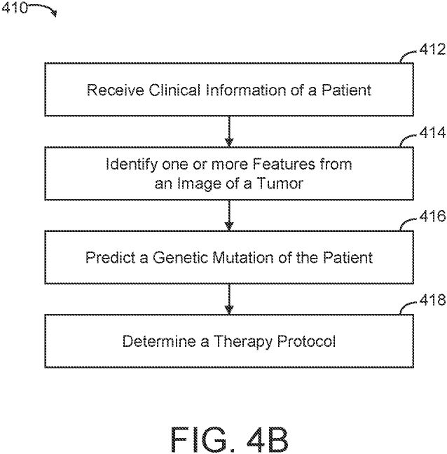

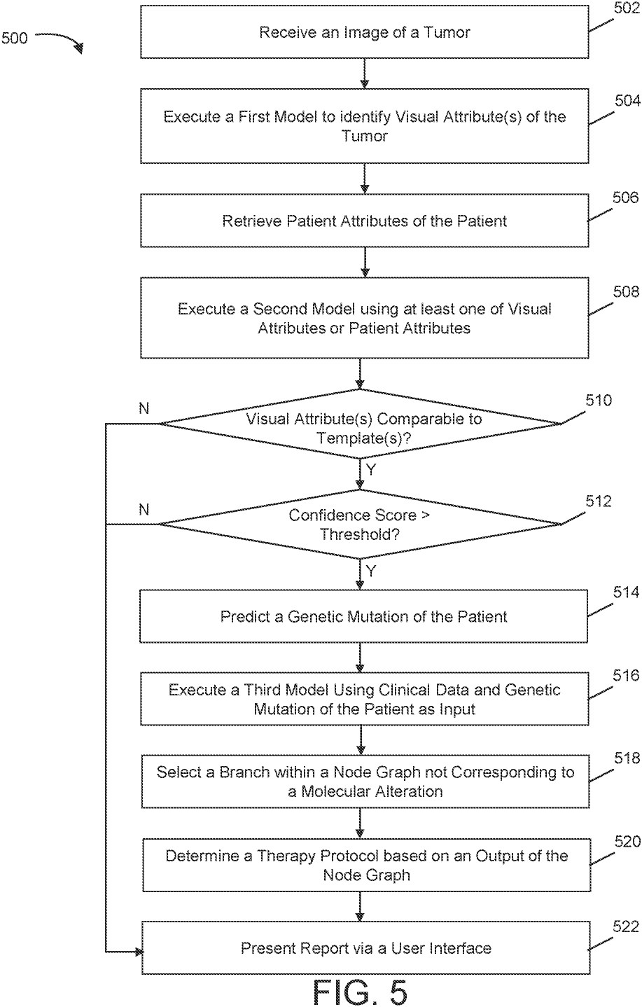

For example, the analytics server 310a can execute at least one of the AI models 360a-c to generate output data. The analytics server 310a can forward the output data to the administrator computing device 350 for the HCP, for example. The analytics server 310a can forward the output data to the end-user devices 340 (e.g., client devices) for the associated patients. As discussed herein, for example, the features or functionalities of the analytics server 310a, the AI models (e.g., first model 360a, second model 360b, or third model 360c), or other devices within the network 330 configured to predict genetic mutation(s), molecular alteration(s), or determine the therapy protocol of patients can be described in conjunction with at least FIG. 4A-5.

The end-user devices 340 may be any computing device comprising a processor and a non-transitory machine-readable storage medium capable of performing the various tasks and processes described herein. Non-limiting examples of an end-user device may include workstation computers, laptop computers, tablet computers, and server computers. In operation, various end-users may use end-user devices 340 to access the platform operationally managed by the analytics server 310a to enter product information and view predicted/projected results.

The administrator computing device 350 may represent a computing device operated by a system administrator. The administrator computing device 350 may be configured to display retrieved data, in the form of results generated by the analytics server 110a, where the system administrator can monitor various models utilized by the analytics server 110a, review feedback, and modify various thresholds/rules described herein. In a non-limiting example, the system administrator may monitor the training of the AI models and/or generation of the training datasets.

The analytics server 310a may access, train, and execute a plurality of AI models. Although the example system 300 depicts the AI models 360a-c stored on the analytics server 310a, the AI models 360a-c may be stored on another device or server (e.g., store locally or in cloud storage). The analytics server 310a may execute the AI models 360a-c in tandem to predict performance metrics for a product.

Using the methods and systems described herein a server (e.g., analytics server 310a), data processing system, or other computing devices can provide disease algorithms that also leverage at least one of the molecular quantitative imaging biomarkers or clinical information of the patients that will help in the prediction of genetic mutations or molecular alterations, such as for the identification of patients for molecular therapy and also identify responders vs. non-responders, for example.

The disease algorithms discussed herein can leverage public datasets (datasets that can include clinical, genomics, histopathological, radionics, and/or digital pathology data) to generate or create algorithms (e.g., models) that will help identify patients with specific molecular alterations leveraging clinical and imaging proxies. These proxies can be generated by AI/ML methods to find the right patients and reduce the overall burden for the HCPs and/or clinical trial investigators. Upon identification of a patient for a molecular therapy, the disease algorithm using systems biology modeling can help identify co-aberrant molecular alterations that could lead to resistance in a specific molecular therapy. This can assist with excluding patients with resistance or recommending patients for one or more therapies or medications.

The disease algorithm can help in identifying the right patient for the right molecular therapy by leveraging clinical and imaging proxies generated using public datasets (from public data sources) or private datasets (e.g., datasets owned by the same entity executing the disease algorithm), thereby reducing the burden (e.g., cost) of performing all the molecular testing. The disease algorithm can be executed to perform the features or functionalities discussed herein to help predict genetic mutations or molecular alterations used to identify the molecular testing to be performed for verification, hence minimizing the number of testing necessary and improving the accuracy of providing the right therapies or medications. Further, within a specific molecular therapy for a patient subtype, the disease algorithm can help in excluding patients with resistance and identifying responders vs. non-responders. This can improve the overall clinical trial success and help in the adoption of the targeted molecular therapies in the clinical setting.

For example, the disease algorithm can correspond to or be implemented via one or more models (e.g., the first model 360a, the second model 360b, and the third model 360c). The first model 360a can be a machine learning model (e.g., a neural network, support vector machine, random forest, etc.). The first model 360a can perform proxy identification (e.g., extract one or more features or visual attributes) of an image. The first model 360a can identify features or visual attributes for the image itself or for a specific area (e.g., lung, brain, etc., of a patient of which the image was take) of the image. For instance, the first model 360a can receive an image of a lung with a tumor as an input. The first model 360a can utilize any suitable feature extraction technique, such as image alignment, object recognition, principal component analysis (PCA), linear discriminant analysis (LDA), etc., to identify visual attributes of the tumor, the lung, or any other aspect of the image.

The second model 360b can identify a genetic defect (e.g., genetic mutation) based on the extracted features and/or the features regarding the patient. The second model 360b can do so usingone or more templates that each contain a list of visual attributes and/or patient attributes and correspond to genetic defect. For instance, the second model 360b can compare the features extracted from the image of the tumor to a set of one or more templates. Each template can include a cluster of features (e.g., from various images) corresponding to at least one of the genetic mutations. The template may be generated or developed using a clustering technique, for instance, by clustering or grouping similar between patients with or images corresponding to a respective genetic mutation. Based on the comparison, the second model 360b can identify or select one of the templates having similar cluster of features compared to the one or more features from the image of the tumor. In this case, the second model 360b can thereby predict the genetic mutation of the patient based on the predefined genetic mutation corresponding to the template, for example.

In some embodiments, the second model 360b is a machine learning model (e.g., a neural network, a support vector machine, a random forest, etc.). The second model 360b can be configured or trained to receive attributes identified from images of tumors and/or patient attributes as input. The analytics server 310a can execute the second model 360b using visual attributes of an image of a tumor of a patient and/or patient attributes (e.g., attributes of a patient, such as age, weight, gender, height, etc. of the patient as well as clinical attributes of the patient, such as values of recent scans or bloodwork that was done on the patient or any notes a doctor has taken regarding the patient, including regarding the tumor depicted in the image) and the second model 360b can output a prediction of identifications of one or more genetic defects.

The third model 360c can include instructions for parsing a nodal graph or biological pathways for therapy protocol selection. For example, the third model 360c can access a database, such as a local or remote database storing the nodal graph including various biological pathways. The nodal graph can be generated by at least one device within the network 330, such as the administrator computing device 350 or the analytics server 310a (e.g., using public data sources to identify the relationships (e.g., mapping) between genetic mutations, molecular alterations, and/or therapy protocol (e.g., medications or therapies)). The device that stores the nodal graph can continuously update the nodal graph by adding or removing different edges and/or nodes to the nodal graph, thus creating different biological pathways within the nodal graph. The device can update the nodal graph as the device receives data for the nodal graph. Each biological pathway can correspond to at least one of the mappings (e.g., genetic mutation(s), molecular alteration(s), or therapy protocol(s)). The third model 360c can receive input from the analytics server 310a indicating at least the genetic mutation(s) and/or the molecular alteration(s) (e.g., criteria or parameters to perform a look-up) associated with the patient. Based on the input criteria, the third model 360c, using the nodal graph, can traverse one or more biological pathways, to determine at least one biological pathway corresponding to the input criteria. Subsequently, the third model 360c can determine/select the biological pathway satisfying the input criteria. The third model 360c can traverse the different biological pathways of the nodal graph (e..g, identify a path of nodes with connecting edges based on the the identified genetic mutation or genetic mutations from the second model 360b and/or other genetic information regarding the patient). The third model 360c can identify the therapy protocol that corresponds to the traversal, which in some cases may correspond to the last node of the traversed biological pathway.

The systems and methods of the technical solution discussed herein can enable the identification of patient cohorts that are resistant to various approved therapies (e.g., avoid therapy protocol resist by the patient), help in new target identification, allow exploration of new pathways that can be considered for multi-resistance clones, or explore combination therapies for co-aberrant gene alterations.

The systems and methods of the technical solution discussed herein can enable the identification of patients for the clinical trials; allow intelligent protocol design with additional inclusion and exclusion criteria; help/assist in the faster and effective screening of patients with minimized physical or monetary burden to the HCPs; or identification of responders vs. non-responders.

The systems and methods of the technical solution discussed herein can provide evidence to physicians for evidence-based clinical guidelines for molecular targeted therapies, define guidelines for permutation-combination without evidence, and/or help in screening the right patients for molecular therapy thereby minimizing the burden on the HCPs.

In various configurations, the systems and methods of the technical solution discussed herein can be implemented within open-source bioinformatics tools, systems biology modeling packages, Bioconductor packages, various biomedical ontologies, such as GO, UNIPROT, etc., or pathways databases, such as KEGG, Reactome, Biocarta etc. These tools or systems can be operated on remote devices or local devices to the systems and methods discussed herein. The tools or systems can be updated periodically or responsive to receiving new patient information (e.g., clinical data, DICOM images, etc.), thereby allowing the tools or systems to be up-to-date for improved accuracy of identifying, predicting, or determining genetic mutations, molecular alterations, or therapy protocols (e.g., medicine or therapy).

As discussed herein, the models, including or corresponding to the disease algorithm, can be generated, trained, or updated using public data sources. The systems and methods can leverage the public data sources for systems biology modeling and semantics/ontology, for instance, to identify clinical and imaging proxies. The proxies can be used as input to further identify genetic mutations and molecular alteration (e.g., resistance) of the patient to include or exclude the patient for a particular therapy protocol. Upon development, the model can be applied to client data (e.g., the client data as input for the model) as a service (deployed on client environment). As the client data may include sensitive drug discovery or trial data, it may be challenging to implement in local environments.

Referring to FIG. 4A, depicted is a flow diagram of an example method 400 for predicting genetic mutation and identifying therapy protocol. The example method 400 can be executed, performed, or otherwise carried out by one or more components of the system 200 (e.g., procedure identification system 202, prediction feed(s) 206, gateway service 208, etc.), the one or more components of the system 300 (e.g., analytics server 310a, system database 310b, electronic data sources 320, end-user device 340, administrator computing device 350, etc.), the computer 100, or any other computing devices described herein in conjunction with FIG. 1A-3. The method 400 can include receiving an image of a tumor of a patient, at operation 402. At operation 404, the method 400 can include executing a first model (e.g., the first model 360a) to identify one or more visual attributes of the tumor. At operation 406, the method 400 can include executing a second model (e.g., the second model 360b) to predict a genetic mutation of the patient. At operation 408, the method 400 can include identifying a therapy protocol associated with the tumor. The operation 408 can include executing a third model (e.g., the third AI model 360c). The various operations discussed herein can be operated by at least one processor of the analytics server 310a, the end-user device 340, or the administrator computing device 350, among other devices authorized to access the patient information. In various implementations, the processor can be coupled to memory (e.g., system database 310b) or configured to access information from the electronic data sources 320, such as public data sources including public datasets.

Still referring to FIG. 4A, and in further detail, at operation 402, the processor can receive an image of a tumor of a patient. The processor can receive the image of a tumor provided by another device, such as the administrator computing device 350 or the end-user device 340. The processor may retrieve the image of the tumor from the local database or memory device (e.g., system database 310b) or from a remote database (e.g., electronic data source 320). The database may include or correspond to an electronic medical record (EMR) system storing various information related to the patients, such as clinical information (e.g., age, gender, demographic information, blood type, etc.), imaging records, previous diagnostic results, etc. The image can include any medical imaging or scan of a patient, such as digital imaging and communications in medicine (DICOM) images, X-ray images, computed tomography (CT) scan images, magnetic resonance imaging (MRI) images, etc. For purposes of providing examples herein, the images may be DICOM images presented in a DICOM viewer (e.g., a user interface capable of or configured to present the DICOM images). Further, for purposes of simplicity, a type of tumor, such as a lung tumor, can be provided as an example herein, although the features or functionalities of the processor (e.g., using one or more models) can be performed on other types of disease, such as brain tumors, breast cancer tumors, throat cancer tumors, etc.

The image can be stored in the local or remote database in at least one of 2D format (e.g., as a collection of pixels) or 3D format (e.g., as a collection of voxels), such that the processor can retrieve the images in 2D or 3D format. In some cases, the processor can retrieve and convert the image from 2D format to 3D format. For example, the processor can retrieve 2D images including at least one of sagittal, coronal, or axial 2D images. The processor can use at least one suitable imaging conversion or synthesis technique to estimate a depth map for the 2D images (e.g., the total depth formed by the images in various orientations) and synthesize the 2D images to form the 3D image of the tumor of the patient, for example. In various cases, the 2D images can be pre-processed or converted to 3D images and stored in the local or remote database.

At operation 404, the processor can execute a model (e.g., a first model) to identify one or more visual attributes of the tumor using the image of the tumor as input. The model can be an AI/ML model (e.g., a neural network, a support vector machine, a random forest, etc.). The visual attributes may include, correspond to, or refer to one or more features (e.g., imaging features) or proxies (e.g., output identifications of features identified based on a probability of the features exceeding a threshold or satisfying another criterion) identified from the image of the tumor. For example, the processor can input the image of the tumor into the model. The processor can execute the model to cause the model to generate probabilities for different proxies or features of the image (e.g., of the tumor depicted in the image). Examples of such proxies include, but are not limited to, emphysema spiculated, lobulated or poorly defined margins, smaller tumors, bubble like lucency, military nodules across the lung (Exon 20), vascular convergence, primary emphysema laterality, centrilobular emphysema pattern, no airway abnormalities, presence of reticulation in nodule, solid nodule attenuation, etc. The processor can compare the probabilities to a threshold and/or each other probability of the output probabilities. The processor can identify the proxies that exceed the threshold and/or the highest defined number of proxies as proxies for the image of the tumor. Accordingly, the model can be configured or trained to identify notable or significant features or proxies for predicting the genetic mutation (associated with the tumor) of the patient. The imaging proxies including the visual attributes can be used to infer (e.g., indicate the likelihood of) a type of genetic mutation that a patient has.

The processor can train the model to identify visual attributes of tumors from images. For example, the processor (or other devices) can generate or train the model using training data associated with patients. The training data can include historical data including various images of tumors of different patients. Each image of a tumor can include or be associated with one or more labels (e.g., ground truth values) or a list of one or more visual attributes (e.g., features or proxies). The model can output identifications of different visual attributes using the images as input. The model can compare the identified visual attributes (e.g., the identifications of the visual attributes) with the labels for each image to determine differences between the output and the labels. The model can use a loss-function and/or backpropagation techniques based on the differences to adjust the internal weights and/or parameters of the model. The model can make such adjustments for each image of the training data set to be trained to accurately output identifications of features or proxies of images or tumors for genetic mutation prediction.

In one example, the processor can obtain historical images of different patients as part of a training dataset. The processor can obtain the historical images or data of the patient from the electronic data source 320, or other data sources (e.g., the cancer genome atlas (TCGA), the cancer imaging archive (TCIA), cBioPortal, EMR, etc.). The historical images can be grouped or clustered into different categories, such as according to the type of tumors (e.g., lung tumor, brain tumor, etc.), genetic mutation(s) for each type of tumor (e.g., one or combinations of types of genetic mutations) of the patient, pre-identified or predetermined labels indicating notable visual attributes or features for the respective tumor (e.g., an indicator of features inferring one or more types of genetic mutations), resistance or molecular alteration of the patient, or other diagnostic information associated with the tumor. Such division can be separated into different buckets, data classification, or datasets (e.g., each dataset including a set of images corresponding to at least one of the different categories).

For respective groups of the tumor type or one or more genetic mutations associated with the tumor, the model can utilize at least one suitable classification technique to, for instance, compare or map similarities or differences between identifiable visual attributes to determine a subset of the visual attributes (e.g., features or patterns) that infer the particular tumor type or genetic mutation(s) for the group. For example, by performing the comparison to identify similarities or differences between identifiable visual attributes (e.g., during operation or training of the model), the model can learn to distinguish between the visual attributes that are relevant or irrelevant to certain types of genetic mutations, such as compared to the template. Hence, the model can be configured or trained to identify one or more visual attributes from images of a tumor, for instance, according to a list of visual attributes that infer or indicate one or more genetic mutations of the patient.

The processor can generate or train the model or receive the model as trained from another computing device. For example, in some cases, the processor can retrieve a trained model from the electronic data source 320, among other data sources. The trained model may be trained by one or more other computing devices within the network 330. The model stored in the electronic data source 320 can be updated by the computing devices via the provision of updated information or data. In some cases, the processor (e.g., of the analytics server 310a) can be configured to generate, train, or update one or more models discussed herein. In some other cases, other devices within the network 330 may be configured or responsible for generating, training, or updating the one or more models. In such cases, the processor can obtain the model from the other devices (e.g., storage device of the other devices). The processor may store or access the one or more models from the local storage device (e.g., system database 310b) or a remote storage device.

To identify or extract one or more visual attributes of a tumor, the processor can acquire an image (e.g., a DICOM image of the tumor with axial, coronal, and sagittal orientations) from an accessible database that stores images of the patient. The processor can pre-process the image using various suitable image pre-processing techniques, such as image normalization, universal orientation to axial format or neuroimaging informatics technology initiative (NIfTI) format, etc. Subsequent to pre-processing the image, the processor can perform mask segmentation on the pre-processed image to identify the tumor and the associated pathology of the tumor, such as the type of tumor that the patient has. For instance, the masking process can include or involve filtering portions of the image considered to be irrelevant to the tumor or extracting portions of the images considered to be relevant to the tumor. The model can consider what portions are considered to be relevant or irrelevant according to the training dataset used for training or updating the model. For instance, the model can be trained using at least one object recognition technique (e.g., supervised or unsupervised training) on sample images to identify areas where the tumor resides, such as the lung, brain, etc. Examples of the visual attributes of images may include tumor, edema, necrosis, indications of ground-glass opacity (GGO), atelectasis, pleural effusion, spiculated signs, or other notable features extracted from the images. Additionally or alternatively, the visual attributes may include measurements or estimates of the density, opacity, size, coloration, depth, etc., associated with each feature.

In various implementations, the visual attributes can include visually identifiable changes in the tumor of a patient over time. For example, the processor, using the model, can compare a first image of the tumor to a second image of the tumor from the same patient captured at different time periods, such as 1 month, 2 months, or 3 months apart. The processor, using the model, can identify whether the tumor is growing, shrinking, spreading, contained in a certain area of the patient body, etc. The visual changes to the tumor can be an indication of at least one particular type of mutation, or otherwise be identified as proxies that can be used to determine a type of mutation of the tumor.

The processor can train, generate, or update the model using any suitable machine learning techniques, image processing techniques, or feature extraction techniques, for example.

At operation 406, the processor can execute a model (e.g., second model) to predict a genetic mutation of the patient using the one or more visual attributes as input. In some cases, the second model can include or be a part of the first model, such that the features discussed herein can be parts of a single model). In some other cases, the second model can be a separate model from the first model.

Similar to the first model, the processor (or other devices within the network 330) can train the second model using historical data from the electronic data sources 320, or other data sources storing historical information related to various types of genetic mutations. For example, the processor can retrieve training datasets including a list of visual attributes (e.g., features or patterns) corresponding to a respective image and one or more genetic mutations identified for the patient associated with the image. The processor can input the training datasets to the model (e.g., the second model) for training or updating the model. The model can group images according to a respective genetic mutation or a certain combination of genetic mutations diagnosed for the patients. In each group of images, the model can compare and contrast visual attributes similar or different between the images to determine one or more visual attributes relevant to a particular genetic mutation or combination of genetic mutations. In some cases, the similarities (or differences) between visual attributes may refer to or include the presence or absence of certain visual attributes. In some cases, the similarities between visual attributes can involve similarities in the values or a range of values for certain measured visual attributes (e.g., gray level, contrast, size, uniformity, etc.). According to the identified similarities or differences between the visual attributes, for example, the model can be trained to identify the indicators/signs (e.g., presence or absence of visual attributes or measurements of visual attributes) of one or more genetic mutations.

The processor can store the trained model in the local database (e.g., memory device or system database 310b). In some aspects, the processor can share or transmit information (e.g., code, script, or program) of the trained model to another device within the network 330, such as the electronic data source 320, the end-user device 340, the administrator computing device 350, etc., for storage or to be executed. In some implementations, the model may be trained by other devices within the network 330. The processor can retrieve and execute the trained model to predict the genetic mutation(s) of the patient.

The processor can use the visual attributes as inputs for the model (e.g., the second model). The model can process the input to identify one or more genetic mutations likely to be associated with the tumor of the patient. In some cases, the model can use templates (e.g., visual attributes identified to be associated with respective one or more genetic mutations) to determine the genetic mutation(s) of the patient. For example, the processor can execute the model to compare the one or more visual attributes to one or more templates. Each template can correspond to a different type of genetic mutation, for instance, including visual attributes historically identified to be relevant to the type of genetic mutation. Responsive to the comparison, the model can determine at least one template having visual attributes that are most similar to the one or more visual attributes of the patient. Accordingly, based on the template, the model can output an identification of at least one type of genetic mutation likely to be associated with the patient.

In some implementations, the model (e.g., the second model) can be a clustering model (e.g., k-means clustering model, a Gaussian mixture model, a balance iterative reducing and clustering using hierarchies (BIRCH) algorithm, a density-based spatial clustering of applications with noise (DBSCAN) model, etc.) The different clusters of the clustering model can each correspond to a different genetic mutation. The processor can generate or train the model by labeling different sets of features or proxies (e.g., features or proxies that can be identified from images, such as images of tumors, and/or features or proxies that can be retrieved from a database, such as data regarding patients that have such tumors). The processor or another device can iteratively generate the clusters by labeling individual sets of data with the ground truth genetic mutation and add the sets of data as individual data points to a graph. Each cluster can be a template. When the processor executes the model using a new set of features or proxies, the model can identify the cluster that is closest to the new set of features or proxies. The identified cluster can be the genetic mutation that corresponds to the patient or tumor.

In some implementations, the processor may retrieve patient attributes of the patient from the database (e.g., electronic data source 320). The patient attributes can correspond to or be a part of the clinical proxies of the patient. The clinical proxies (e.g., patient attributes) can include clinical information historically assessed or diagnosed for the patient. For example, the clinical proxies can include background or general information regarding the patient, such as gender, age, ethnicity, blood type, whether the patient smokes, etc. Additionally or alternatively, the clinical proxies can include specific information regarding the tumor of the patient, historically diagnosed or assessed by the HCP, such as pathological stage, histological subtype, metastasis, progression pattern, tumor grade, recurrence, tumor location, etc.

In some cases, the clinical proxies can include information regarding the genetic mutation of the patient historically tested by the HCP or predicted by the model (e.g., the second model). In this case, the processor may execute the model using the imaging proxies (e.g., including the one or more visual attributes) and the clinical proxies as inputs. For example, the model can be trained using visual attributes of patients and the clinical information of the respective patients. Given the certain types of genetic mutation (and in some cases genetic alterations, as discussed herein), the model can be trained to identify the visual attributes and patient information (e.g., smoker vs non-smoker, frequency of smoking, blood pressure, blood type, information from patient’s blood test, etc.) relevant to or inferring the given genetic mutation.

After providing the model with the inputs, the processor can execute the model to leverage the clinical proxies for increased accuracy in identifying the one or more genetic mutations of the patient. In some cases, the clinical proxies can include an indication of the type of genetic mutation previously assessed by the HCP or predicted by the model (and validated by conducting the corresponding genetic test(s)). In this case, the model (e.g., second model) may identify or select the visual attributes associated with the type of genetic mutation indicated in the clinical proxies and compare the identified visual attributes to the visual attributes extracted from the image of the tumor of the patient. In this case, by comparing the visual attributes expected to be presented based on the previously diagnosed type of genetic mutation to the visual attributes of the present image, the model can confirm whether the diagnosed type of genetic mutation is still present for the patient (compared to a previous prediction of a genetic mutation). The model may also identify any other visual attributes that potentially indicate at least one other type of genetic mutation, such as based on the extracted visual attributes from the present image.

In some configurations, the model can use the one or more visual attributes and the clinical proxies (e.g., patient attributes) to determine a confidence score (e.g., likelihood score) for different types of genetic mutations. If the clinical proxies include a particular type of genetic mutation previously diagnosed for the patient and the visual attribute(s) infer the same type of genetic mutation, the model can provide a relatively high confidence score, such as 80%, 90%, etc. For instance, the more closely aligned the one or more visual attributes are to the template (e.g., indicating visual attributes corresponding to one or more types of genetic mutations), such as the number of visual templates that match the template, the higher the confidence score or vice versa. By matching (or identifying similarities or differences between) the one or more visual attributes and the template, the model can estimate or determine the confidence score for each of the predicted type of genetic mutations.

For example, the model (e.g., clustering model) can cluster or group the visual attributes extracted from the images of the tumor. The model can perform the clustering using at least one suitable clustering technique, such as centroid-based clustering, k-means clustering, hierarchical clustering, density-based clustering, etc. The clusters can be a part of the templates corresponding to at least one type of genetic mutation. For instance, each cluster of visual attributes can include a centroid (e.g., the center of the cluster) indicating an average for the respective visual attribute to infer the type of genetic mutation. In this case, the model can compare the distance between the point of input proxies (e.g., imaging proxies including the visual attributes and/or the clinical proxies including patient attributes) and the centroid (e.g., the center) of the closet cluster associated with at least one template. Based on the comparison, the model can identify one or more clusters closest to the input proxies according to the centroids of the clusters. Subsequently, the model can identify the template corresponding to the clusters closest to the input proxies and predict at least one type of genetic mutation corresponding to the template.

In some cases, the model can determine the confidence score of the inferred genetic mutation(s) based on specific information related to the tumor from the patient attributes, such as pathological stage, historical subtype, metastasis, etc. In some other cases, the model can determine the confidence score of the inferred genetic mutation(s) according to the basic or general information of the patient, such as whether the patient is a smoker, drinks alcohol, the cholesterol level, blood pressure, etc., which may increase or decrease the likeliness of the patient having certain genetic mutations in regards to the tumor, for example. After the determination, the processor (using the model) can select the genetic mutation(s) based on the confidence score. An identification of the selected genetic mutation(s) can be the output by the model (e.g., the second model).

During training, the processor can perform feature selection techniques to determine which proxies to use as input into the model (e.g., the second model). Feature selection techniques can involve identifying attributes or proxies that have the highest impact, or any impact at all, in predicting genetic mutations of patients. The processor can identify the attributes or proxies that have the highest impact, for example, by identifying features that are common across different templates or that otherwise do not have a correlation (e.g., a statistical correlation) with any particular templates. The processor can use feature selection techniques such as categorical input, categorical output techniques (e.g., chi-squared test or mutual information) to identify features to filter out before inputting the features into the second model. Accordingly, when executing the second model with proxies, the processor can discard (e.g., remove) or otherwise not include clinical proxies the processor retrieved from a database regarding and/or visual proxies the first model identified from an image of a tumor of the patient in the input to the second model.

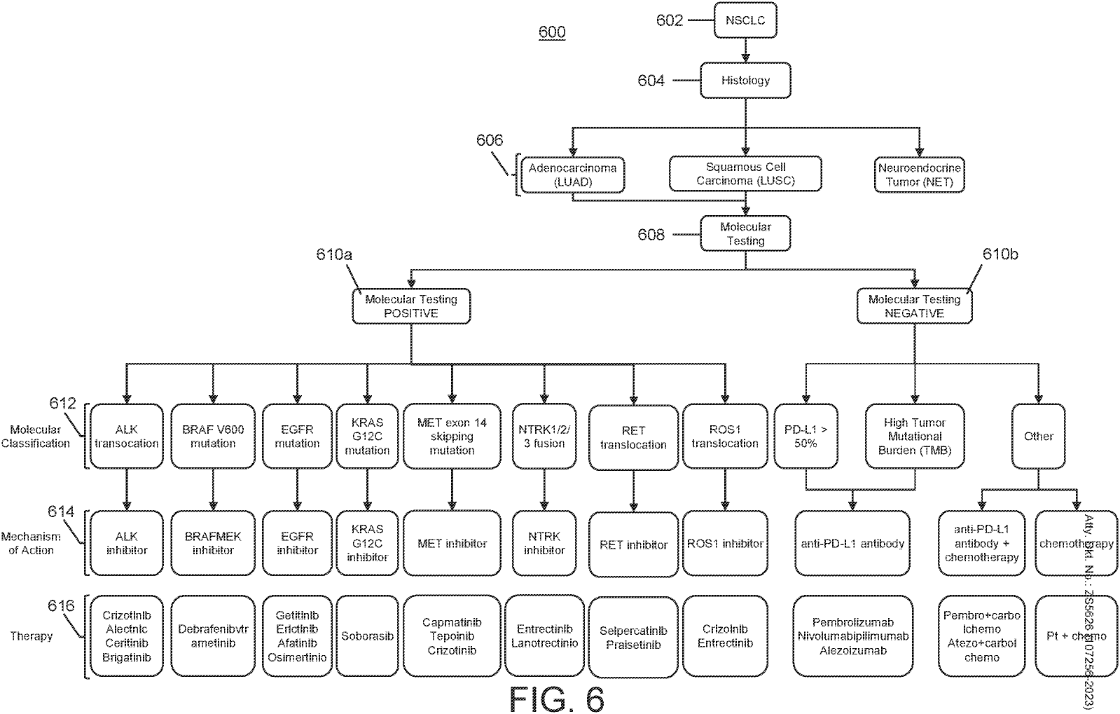

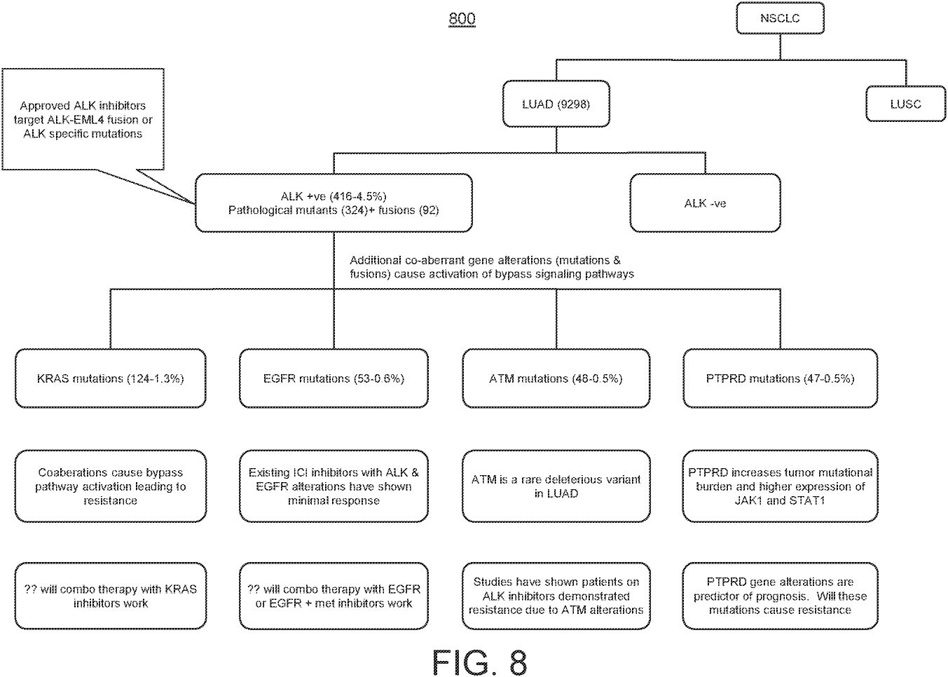

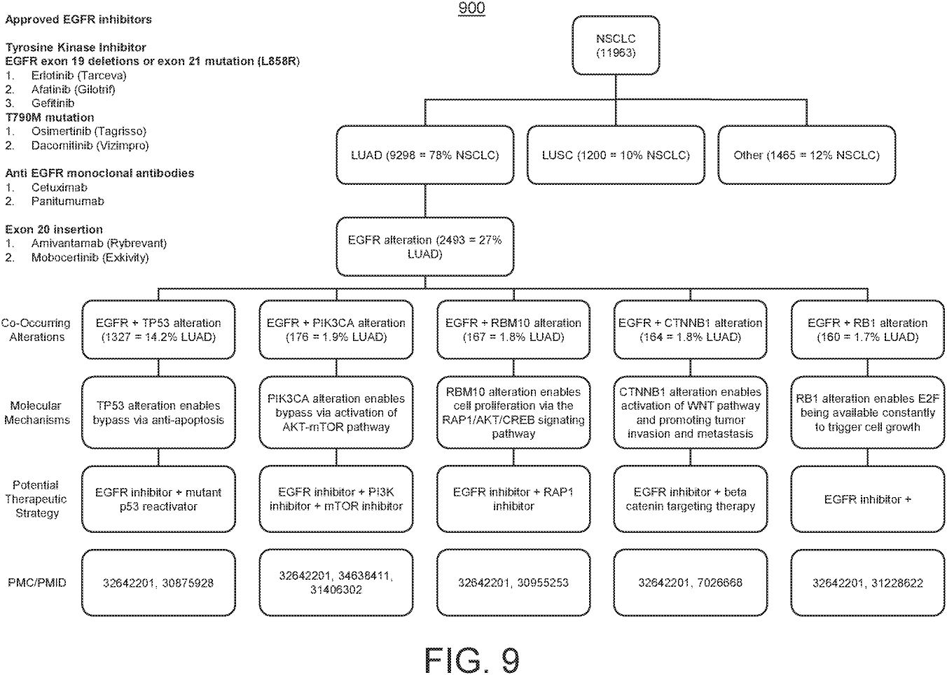

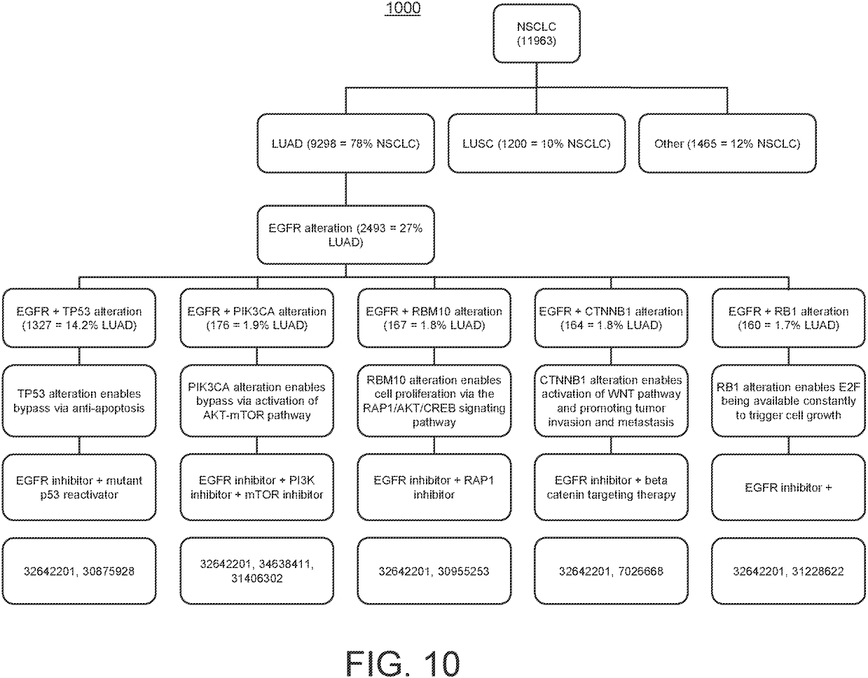

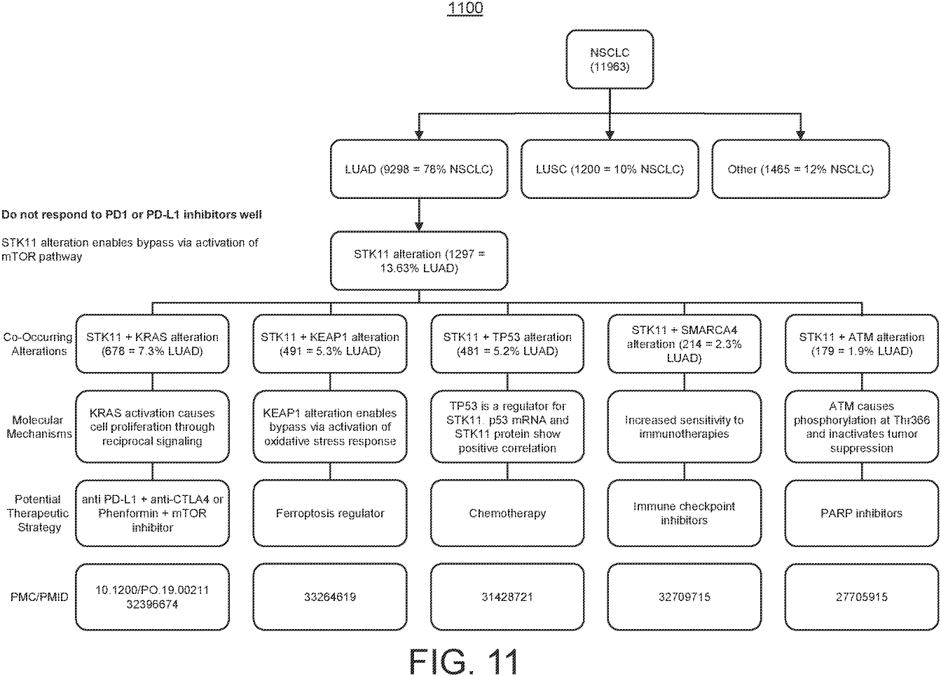

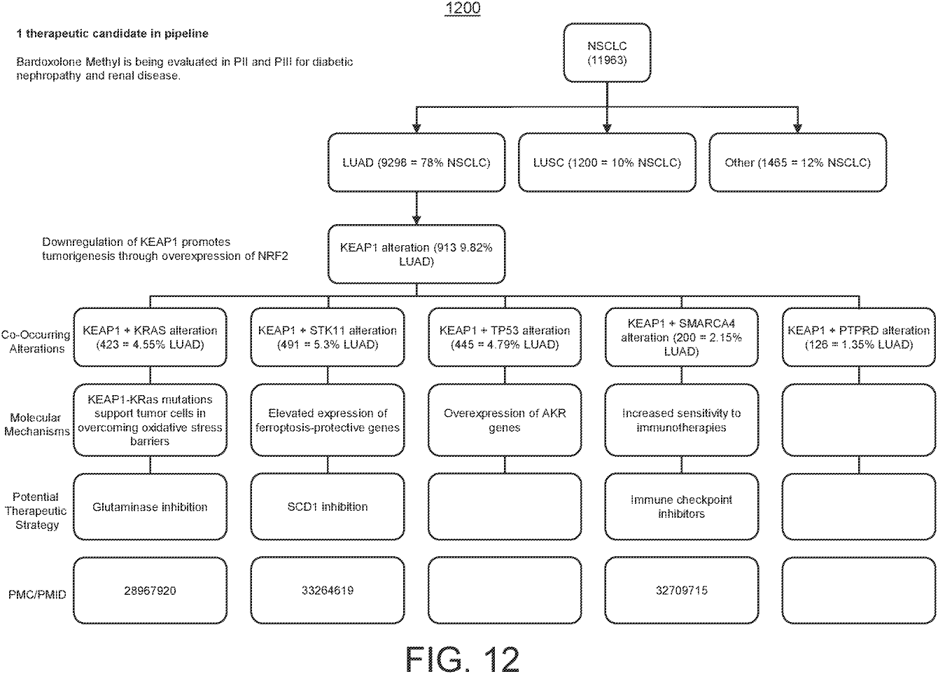

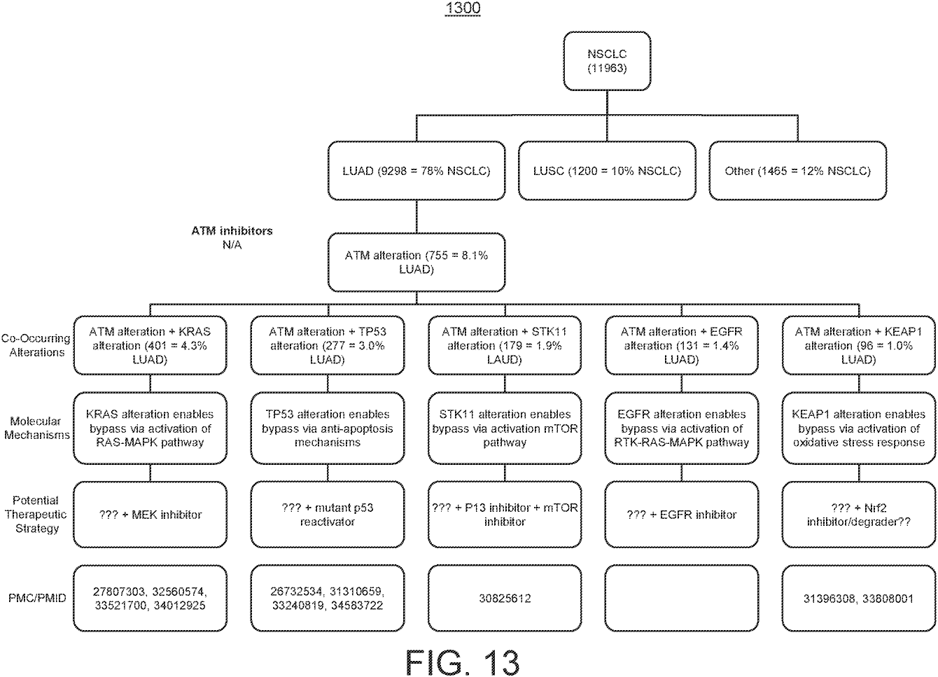

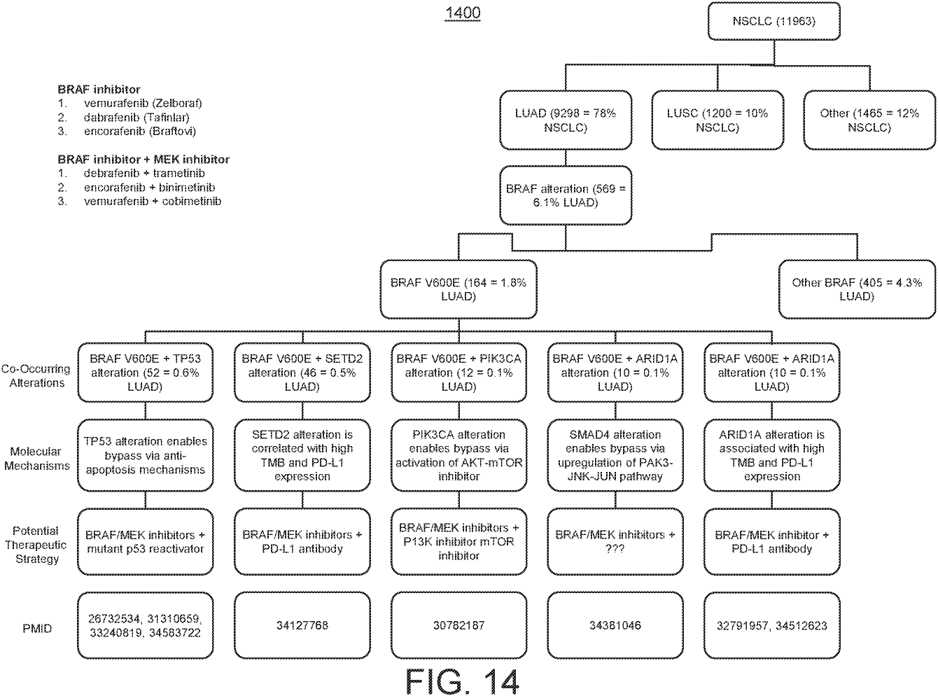

At operation 408, the processor can identify a therapy protocol associated with the tumor based on the genetic mutation. The therapy protocol may include or correspond to at least one of the therapeutic strategy (e.g., inhibitor or medication to prescribe) or the type of therapy to be performed (e.g., targeted therapy, radiation therapy, surgery, etc.). The therapy protocol can be predetermined for a particular type of genetic mutation. For example, an anaplastic lymphoma kinase (ALK) translocation can be associated with at least one of ALK inhibitor, crizotinib, alectinib, ceritinib, or brigatinib, among others. In another example, an epidermal growth factor receptor (EGFR) mutation can be associated with at least one of EGFR inhibitor, gefitinib, erlotinib, afatinib, or osimertinib, among others. Other types of genetic mutations can be associated with predetermined therapy protocol, such as described in conjunction with at least one of FIGS. 6-14.

In some cases, the processor can identify or select the therapy protocol according to the stage or severity of the tumor (e.g., based on at least one of the clinical proxies or imaging proxies). For instance, the processor can select different checkpoint inhibitors for a certain type of genetic mutation and/or molecular alterations to regulate the tumor at different stages. The checkpoint inhibitors can be considered when the patient is negative (e.g., according to the clinical proxies and/or imaging proxies) for molecular alterations and/or genetic mutations. The checkpoint inhibitors can be used to target checkpoints on immune cells (e.g., PD-L1, etc.) or tumor cells (e.g., PD-1, CTLA4, etc.). Hence, the processor can identify at least one checkpoint inhibitor appropriate for the particular genetic mutation(s).

In some implementations, to identify the therapy protocol, the processor may determine the molecular alteration (e.g., resistance) of/for the patient. The processor may execute a model (e.g., a third model, such as the third AI model 360c) using the one or more patient attributes (e.g., clinical proxies) as input to predict the molecular alteration. In some cases, the third model can correspond to or be a part of the first model or the second model, such that a single model can perform the features or functionalities of the AI model discussed herein. In some other cases, the third model can be a separate model from the first model or the second model.

In some implementations, the third model can be generated, trained, or updated similarly to the first model or the second model. For example, the processor can provide training datasets to the model including at least historical clinical information of patients and classified/determined molecular alteration(s) for the respective patients. The model can identify, according to the training datasets, types of clinical information relevant for indicating at least one molecular alteration. In some cases, the relevancy of the clinical information to certain types of molecular alterations can be provided in the training datasets. The processor may input sample datasets for training or validation of the model, using at least one suitable machine learning technique, such as regression techniques, association techniques, etc. In some cases, the model can be executed using rule-based algorithms or functions, for instance, by traversing the pathways of a nodal graph according to different parameters or criteria (e.g., types of genetic mutations and/or molecular alterations). In various aspects, the one or more models discussed herein may be trained using similar, additional, or alternative techniques/methods.

Based on the molecular alteration(s) of the patient (e.g., types of molecules the patient is resistant to), the processor can determine the therapy protocol for the patient. For example, the molecular alterations can indicate the resistance of the patient to a certain therapy protocol, such as the initially identified therapy protocol. In this case, the model (e.g., the third model) can be a nodal data structure (e.g., tree data structure) including biological pathways (sometimes referred to generally as pathways) and at least one modality (e.g., therapy protocol) associated with each biological pathway. The processor can obtain the nodal data structure from the local memory or remote database updated using any available clinical information of patients from various databases (e.g., public data sources, electronic data source 320, or other sources). The processor or any authorized devices (e.g., HCP devices or administrator computing device 350) within the network 330 may access or update the nodal data structure with new, improved, or alternative information regarding therapy protocols associated with different types of genetic mutations or molecular alterations.

The processor can traverse the nodal data structure (e.g., using the third model) to determine the therapy protocol. For example, the processor can input the genetic mutation of the patient into the third model to search the nodal data structure. Based at least on the genetic mutation, the model can determine one or more pathways associated with the genetic mutation, where each pathway can be associated with a therapy protocol. In further example, the processor can use the molecular alteration as input to the model. Given the one or more pathways, the model can determine a subset of pathways not impacted or unaffected by (e.g., that doesn’t correspond) the resistance (e.g., molecular alteration) of the patient. In some cases (e.g., when the patient is negative for molecular alterations and genetic mutations), the processor can use checkpoint inhibitors to search the nodal data structure or otherwise select one or more paths of the nodal data structure. Hence, the subset of pathways can include at least one therapy protocol likely suitable for the patient with the genetic mutation and molecular alteration.

Different genetic mutations or combinations of genetic mutations, as well as molecular alteration(s), can lead to different pathways in the nodal data structure. The end of each pathway can correspond to a different therapy protocol. For example, the processor, using the model, can identify a first therapy protocol according to the predicted genetic mutation of the patient. According to the molecular alteration, the processor may determine that the patient is resistant to the first therapy protocol (or one or more other therapy protocols) associated with one of the biological pathways (e.g., the first biological pathway). Subsequently, the processor can identify a second therapy protocol associated with another (e.g., second) biological pathway satisfying the genetic mutation and the molecular alteration predicted for the patient. In another example, if the second biological pathway of the second therapy protocol corresponds to the resistance (e.g., molecular alteration) of the patient, the processor executing the model (e.g., the third model) can avoid this second biological pathway and traverse the first biological pathway to identify the first therapy protocol, for example.

The processor can add or include an identification of the patient to a list of patients for the therapy protocol identified for the patient. Each patient can be associated with a respective identification, such as an electronic identifier (e.g., tag) assigned to a respective patient. For example, after identifying the therapy protocol for the patient, the processor can add the identification of the patient to a list of patients to be tested for the particular mutation or molecular alteration for verification purposes. The patient can be treated using the therapy protocol, in some cases after verifying that the patient has the genetic mutation or molecular alteration associated with the tumor. By performing the implementations discussed herein, the systems and methods of the technical solution can reduce the number of molecular tests necessary to identify mutations or alterations for patients to provide proper therapy protocol, thereby minimizing the burden on the HCPs, while optimizing diagnostic accuracy.

FIG. 4B is a flow diagram of another example method 410 for predicting genetic mutation and determining therapy protocol. The example method 410 can be executed, performed, or otherwise carried out by one or more components of the system 200 (e.g., procedure identification system 202, prediction feed(s) 206, gateway service 208, etc.), the one or more components of the system 300 (e.g., analytics server 310a, system database 310b, electronic data sources 320, end-user device 340, administrator computing device 350, etc.), the computer 100, or any other computing devices described herein in conjunction with FIG. 1A-3. The example method 410 may be performed additionally or alternatively to method 400, as described in conjunction with FIG. 4A. The method 410 can include receiving clinical information of a patient, at operation 412. At operation 414, the method 410 can include identifying one or more features from an image of a tumor. At operation 416, the method 410 can include predicting a genetic mutation of the patient. At operation 418, the method 410 can include determining a therapy protocol. The various operations discussed herein can be operated by at least one processor of the analytics server 310a, the end-user device 340, or the administrator computing device 350, among other devices authorized to access the patient information. In various implementations, the processor can be coupled to memory (e.g., system database 310b) or configured to access information from the electronic data sources 320, such as public data sources including public datasets.

Still referring to FIG. 4B, and in further detail, at operation 412, the processor can receive clinical information of a patient. The clinical information can be a part of the clinical proxies, such as described in conjunction with FIG. 4A. At operation 414, the processor can identify one or more features from an image (e.g., of a tumor or other parts of the image) of the patient. For example, the processor can receive or obtain the image from another device within the network 330, such as the electronic data source 320, end-user device 340, or administrator computing device 350. The processor can use the image as input to a model (e.g., the first model) to identify the one or more features (e.g., visual attributes) of the tumor. In some cases, the model can identify other features in the image that are not part of the tumor. For instance, the model can identify features in different parts of the lung, which may be compared with features of the template to predict the type of genetic mutation(s), for example. The one or more features can be a part of the imaging proxies, such as described in conjunction with FIG. 4A.

At operation 416, the processor can predict, using a model (e.g., the second model), a genetic mutation of the patient according to the one or more features. The processor can use the one or more features as input to the model. Upon execution, the model can output an identification of the genetic mutation inferred by the one or more features. In some cases, the model can output identifications of multiple genetic mutations inferred by the one or more features.

At operation 418, the processor can determine a therapy protocol according to at least one of the clinical information or the at least one genetic mutation of the patient. For example, the processor can obtain or access the nodal data structure from the electronic data source 320, the local database (e.g., system database 310b of the analytics server 310a), or another data source. The processor can use the genetic mutation as input to identify biological pathways impacted or associated with the genetic mutation. Each biological pathway can be associated with at least one respective therapy protocol.

In some configurations, the processor can use the clinical information as input to a model to determine the molecular alteration for the patient. The processor can use the molecular alteration (e.g., clinical information, such as genetic information regarding the patient) in the nodal data structure to identify a subset of the biological pathways that are not affected by the molecular alteration (e.g., branch or pathway not affected or impacted by the resistance of the patient). By using the genetic mutation and the clinical information (e.g., molecular alteration), the processor can identify one or more biological pathways associated with the genetic mutation, including therapy protocol(s) not resistant to by the patient.

In some implementations, the processor can traverse the nodal data structure using multiple genetic mutations predicted by the model, including a first genetic mutation and a second genetic mutation of the patient. The processor can identify one or more biological pathways associated with the combination of genetic mutations. The processor can further identify a subset of the one or more biological pathways according to the clinical information (e.g., molecular alteration) of the patient. Based on the subset of biological pathways, the processor can determine at least one suitable therapy protocol associated with the particular biological pathway.

In some configurations, the processor can be configured to output an identification of a predefined molecular pathway depending on the number of genetic mutations predicted by the model. For example, the processor can be provided or configured (e.g., by the administrator computing device 350 or other authorized devices in the network 330) to provide a predefined type of therapy protocol if the number of predicted genetic mutations is at or above a threshold. Taking a threshold of three, for example, if the processor executing the model (e.g., the second model) predicts more than two different types of genetic mutations according to the one or more features, the processor can determine a predefined therapy to be the therapy protocol according to the number of genetic mutations associated with the tumor of the patient. The predefined therapy protocol may be, for example, chemotherapy, radiation therapy, pembrolizumab, nivolumab, atezolizumab, etc.

FIG. 5 is a flow diagram of an example detailed method 500 for predicting genetic mutation and determining therapy protocol. The example method 500 can be executed, performed, or otherwise carried out by one or more components of the system 200 (e.g., procedure identification system 202, prediction feed(s) 206, gateway service 208, etc.), the one or more components of the system 300 (e.g., analytics server 310a, system database 310b, electronic data sources 320, end-user device 340, administrator computing device 350, etc.), the computer 100, or any other computing devices described herein in conjunction with FIG. 1A-3. The example method 500 may be performed additionally or alternatively to at least method 400 or method 410, as described in conjunction with FIGS. 4A-B.

The method 500 can include receiving an image of a tumor, at operation 502. At operation 504, the method 500 can include executing a first model to identify one or more visual attributes of the tumor. At operation 506, the method 500 can include retrieving patient attributes of the patient. At operation 508, the method 500 can include executing a second model using at least one of visual attributes or patient attributes. At operation 510, the method 500 can include determining whether the one or more visual attributes are comparable to one or more templates. At operation 512, the method 500 can include determining whether a confidence score is greater than (or equal to) a threshold. At operation 514, the method 500 can include predicting a genetic mutation of the patient. At operation 516, the method 500 can include executing a third model using clinical data and genetic mutation of the patient as input. At operation 518, the method 500 can include selecting a branch within a node graph not corresponding to a molecular alteration. At operation 520, the method 500 can include determining a therapy protocol based on or according to an output of the model that traverses the node graph. At operation 522, the method 500 can include presenting a report via a user interface. The various operations discussed herein can be operated by at least one processor of the analytics server 310a, the end-user device 340, or the administrator computing device 350, among other devices authorized to access the patient information. In various implementations, the processor can be coupled to memory (e.g., system database 310b) or configured to access information from the electronic data sources 320, such as public data sources including public datasets.

Still referring to FIG. 5, and in further detail, at operation 502, the processor can receive an image of a tumor of a patient. At operation 504, the processor can execute a first model to identify one or more visual attributes (e.g., one or more features) of the tumor or the image. The processor can use the image as input into the first model and execute the first model. The first model can output identifications of one or more visual attributes of the tumor or the image based on the execution.

At operation 506, the processor can retrieve or obtain patient attributes (e.g., clinical information) of the patient. The processor can retrieve the patient attributes from a database, such as a local database, remote database, or other data repositories accessible via the network 330. The patient attributes or clinical information of the patient can correspond to clinical proxies for the patient.

At operation 508, the processor can execute a second model using at least one of the identified visual attributes or patient attributes as input. The second model can be configured to predict one or more genetic mutations associated with the patient based on visual attributes and/or patient attributes. To perform the prediction, the second model can compare the visual attributes and/or the patient attributes to one or more templates (e.g., clusters). For example, each template can include a list of visual attributes or patient attributes of the template. Each template, can be associated with at least one genetic mutation.

At operation 510, the processor can determine whether the one or more visual attributes are comparable to one or more templates. If the one or more visual attributes or patient attributes are comparable to at least one of the templates (e.g., has a matching number or attributes to at least one of the templates above a threshold), the processor can generate a list of one or more potential genetic mutations that correspond to the matching templates. The processor can proceed to operation 512 to select, for instance, one or more genetic mutations likely to be associated with the patient. If there is no comparable template to the visual attributes identified by the first model or patient attributes retrieved by the second model, the processor may proceed to operation 522, for instance, to generate a report indicating failure to predict the genetic mutation (or no indication of genetic mutation) for the patient.

In some implementations, the patient attributes can include an indication of at least one type of genetic mutation previously or historically diagnosed for the patient. In such cases, the processor can use the clinical information to determine at least one type of genetic mutation associated with the patient, such that the processor executing the second model can identify any other potential genetic mutation according to the visual attributes of the tumor.

In some cases, instead of comparing the visual attributes to the template(s), the processor may execute the second model to predict one or more genetic mutations based on predetermined criteria or parameters. For example, the second model can compare the visual attributes (e.g., the presence or absence of the visual attributes or values corresponding to the visual attributes) to various sets of criteria. Each set of criteria can correspond to at least one genetic mutation. Each set of criteria can also include a list of visual attributes (or the values of the visual attributes thereof) inferring the at least one genetic mutation. Based on the one or more visual attributes of the image satisfying the criteria predefined for the particular one or more genetic mutations, the processor can predict the inferred genetic mutation(s) for the patient.

At operation 512, the processor can determine whether a confidence score is greater than (or equal to) a threshold. In various implementations, the processor can determine or estimate the confidence score associated with the predicted genetic mutation(s) according to the clinical information or patient attributes (e.g., clinical proxies) and the visual attributes (e.g., imaging proxies) of the patient. The biological information, chemical information, family history, or other health-related information of the patient can be indicators for certain genetic mutations exhibited in the patient with the tumor. For example, the processor can execute the second model to predict one or more genetic mutations for the patient according to the imaging proxies and the clinical proxies. The processor can execute the second model using the proxies as input for comparison with at least one cluster (e.g., of visual attributes, patient information, etc.). Based on the distance between the proxies and the centroid of the cluster(s) being within a predetermined threshold (e.g., within 5% difference in values), the processor can determine that the proxies are similar to the cluster corresponding to at least one respective type of genetic mutation, hence provided with a relatively higher confidence score. Proxies that are relatively farther from the centroid may be provided with a relatively lower confidence score. In some cases, the processor can execute the second model to determine the number of proxies that match the clusters. Based on at least a predetermined number of proxies matching the clusters, the processor can determine the type of genetic mutation according to the cluster or the number of matching proxies. The processor can determine multiple genetic mutations when the proxies are within a predetermined threhsold of multiple centroids, in some cases. The processor may pick the genetic mutation(s) having a relatively high confidence score (e.g., greater than or equal to a predetermined threshold), such as greater than or equal to 65%, 70%, 80%, 90%, etc., based on the configuration of the processor (or the analytics server 310a). If the processor assigned at least one genetic mutation with a confidence score greater than or equal to the predetermined threshold, the processor may select the at least one genetic mutation and proceed to operation 514.

Otherwise, if none of the predicted mutations (e.g., by the second model) is at or above the predetermined threshold, the processor can proceed to operation 522 to report the output or results of the second model to at least one of the end-user of the administrator, such as via end-user device 340 or the administrator computing device 350. For instance, when proceeding to operation 522, the processor may generate a report indicating that no particular genetic mutation has relatively enough confidence score to be selected or inferred to the patient.

At operation 514, the processor can predict a genetic mutation of the patient. The prediction of the genetic mutation can be the output of the second model responsive to the execution by the processor using at least one of the visual attributes or the clinical information of the patient as input.

At operation 516, the processor can execute a third model using clinical data and the genetic mutation of the patient as input. In various implementations, the processor can execute the third model to traverse the nodal data structure to identify at least one therapy protocol according to the genetic mutation and the clinical data/information of the patient. The third model can be configured to traverse the nodal data structure to avoid a therapy protocol for which the patient has a molecular alteration that causes a resistance to the therapy protocol. For example, the third model can traverse the nodal data structure based on different genes and/or gene mutations that the processor identified for a patient. In doing so, the third model can select a path that branches to a specific therapy for which the patient does not have any resistance, in some cases selecting branches instead of other branches that correspond to a resistance.

For example, at operation 518, the processor, executing the third model, can select a branch (e.g., pathway) within a node graph (e.g., nodal data structure) not corresponding to a molecular alteration. For example, the processor can select one or more pathways or branches of the nodal graph associated with the predicted genetic mutation(s) of the patient. Each branch can correspond to a respective therapy protocol for diagnosing the particular genetic mutation or a combination of genetic mutations of the tumor. In some situations, certain therapy protocols (e.g., branches) may not be compatible with some molecular alterations, such that the processor may avoid the branches according to the molecular alteration of the patient.

At operation 520, the processor can determine a therapy protocol based on or according to an output of the model that traverses the nodal data structure (e.g., the node graph). For example, using the genetic mutation(s) and the molecular alteration(s) predicted or determined for the patient, the processor can select a branch from the node graph. Based on the selected branch, the processor can identify the therapy protocol corresponding to the branch.

At operation 522, the processor can present or provide a report, such as to the patient or HCP, via a user interface. The user interface can include or correspond to a graphical user interface for end users or HCP to view the report via a respective device (e.g., end-user device 340 or administrator computing device 350). For example, the processor can generate a report including any diagnostic-related information pertaining to the tumor of the patient. The report may include at least one of, but is not limited to, the clinical proxies, imaging proxies, predicted genetic mutation(s), determined molecular alteration(s), identified therapy protocol accounting for the genetic mutation(s) and the molecular alteration(s), etc. The processor can send the report to the administrator computing device 350, the end-user device 340, or other devices authorized to access the report, such as devices accessed by the HCP or the patient. The processor can store the generated report in the local database or remote database.

In some cases, there may be multiple pathways of the nodal data structure compatible with the specific genetic mutation(s) and molecular alteration(s) for the patient. In this case, the processor may select multiple pathways, thereby identifying multiple therapy protocols for the patient. The processor may generate the report including multiple therapy protocols for selection by the HCP or the patient.

In some implementations, if the visual attributes of the image of the tumor and/or the patient attributes are not comparable to the template or do not indicate a particular genetic mutation, the processor can include in the report that no specific genetic mutation could be found. In some cases, the processor may flag certain visual attributes or clinical information potentially relevant to the prediction of the molecular mutation for the HCP. In some other implementations, if the confidence scores associated with all types of genetic mutations are less than the predetermined threshold, the processor may include in the report that no conclusive prediction of genetic mutation is determined. In some cases, the processor may include in the report the few genetic mutations with the highest confidence scores associated with the patient, such as for consideration by the HCP to further analyze.

Taking a patient with breast cancer, for example, the processor can receive at least one image (e.g., CT image) of the patient. The processor can execute the first model to identify or extract one or more features of the breast cancer. Using the extracted features, the processor can execute the second model to determine the type of genetic mutation, e.g., by comparing the features to one or more templates corresponding to respective types of genetic mutations for breast cancers. In some implementations, the second model may output one or more confidence scores for respective types of genetic mutations, such as a BRAC1 breast cancer with a likelihood or confidence score of 80% and BRAC2 breast cancer with a likelihood of 40%. The predetermined threshold for predicting a certain type of genetic mutation may be at least 60%, 70%, etc. In this example, according to the output of the second model, the processor may predict that the genetic mutation of the breast cancer for the patient is BRAC1. Accordingly, the processor can transmit a notification to the HCP (e.g., to the administrator computing device 350), such that the patient can be tested for that specific type of breast cancer.

Additionally or alternatively from the above examples, the processor can execute the third model, using the clinical information of the patient and/or the predicted genetic mutation as input, to determine any molecular alteration for the patient. Based on the molecular alteration, the processor can determine the resistance the patient has towards one or more therapy protocols. By executing the third model, the processor can traverse the nodal graph to identify the therapy protocol (e.g., drug/medication and/or suitable clinical trial) for the patient suitable for the type of genetic mutation of the breast cancer and not resistant by the patient.

FIG. 6 illustrates an example guideline 600 for molecular tests associated with respective medicines and therapies. The guideline 600 can include or be a part of the nodal data structure for determining the therapy protocol for the patient based on at least the genetic mutation of the tumor. The guideline 600 can represent the steps in identifying the therapy protocol based on, for example, the type of tumor determined for the patient, the classification of the type of tumor, and some potential genetic mutations for the type of tumor. In some implementations, the guideline 600 may be a part of the historical information presenting the relationships or links between the type of tumor, classification of tumor, potential genetic mutations, and related therapy protocols for the genetic mutations. In some other implementations, the processor can use the guideline 600, in part, to identify the therapy protocol after predicting the genetic mutation for a particular type (or classification) of tumor.

For example, the guideline 600 can be represented for a specific type of tumor, such as non-small cell lung cancer (NSCLC) in this case (as shown at 602), although other types of tumors or diseases can be indicated in other guidelines. At 604, histology can be performed on the images or clinical information of the patient to classify the tumor into at least one subtype, such as one of adenocarcinoma, squamous cell carcinoma, or neuroendocrine tumor (shown at 606), in this case. At 608, molecular testing can be performed for the patient to identify or verify the genetic mutation(s) for the patient.

In some cases, at 610a, the molecular testing can be positive (e.g., the test outputs at least one genetic mutation identified for the tumor). In some other cases, at 610b, the molecular testing may be negative, indicating that the test either fail to identify any genetic mutation for the tumor or more than a predefined number of genetic mutations (e.g., more than two) were potentially identified.

According to whether the molecular testing is positive or negative, at 612, the guideline 600 can indicate some of the types of genetic mutations possible for the tumor. The guideline 600 may further indicate the mechanism of action (at 614) or therapy (at 616) that can be recommended (or verified) for the patient to potentially treat the specific tumor. The mechanism of action (e.g., medicine, inhibitor, or antibody, to name a few) and the therapy (e.g., different types of therapies to recommend) may be a part of the therapy protocol.

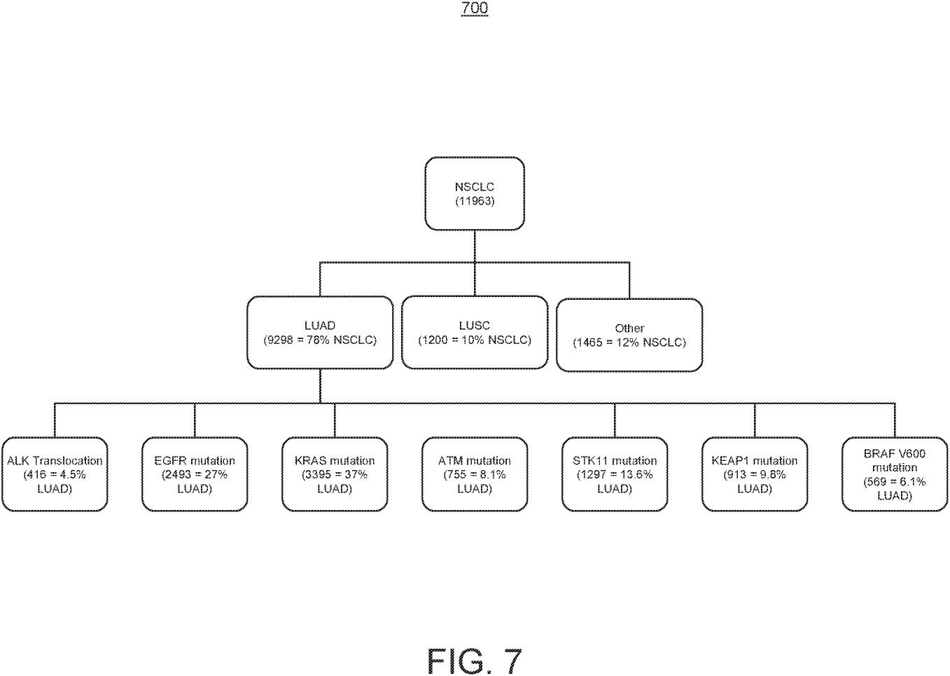

Referring to FIGS. 7-14, illustrate examples of some key oncogenic mutations of a particular disease. Throughout the diagrams 700-1400 of FIGS. 7-14, the NSCLC may be used as an example of the type of tumor of the patient, although other types of tumors may be presented in other examples herein. For this type of tumor, the key oncogenic mutations associated with the respective subtype of the tumor may be provided in further detail.

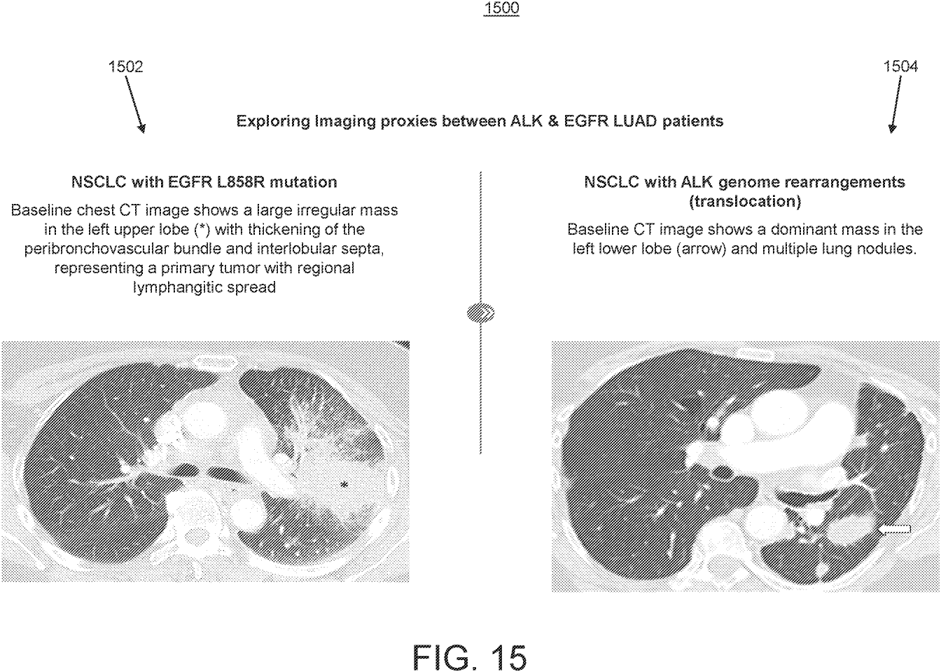

Referring to FIG. 15, depicted is an example comparison 1500 of genetic mutations between different patients. The comparison 1500 can be between the NSCLC with epidermal growth factor receptor (EGFR) L858R mutation (image 1502) and NSCLC with anaplastic lymphoma kinase (ALK) genome rearrangements (translocation) (image 1504). These images 1502, 1504 can be obtained by the analytics server 310a from a data source, such as the electronic data source 320, EMR, etc. Each of the images 1502, 1504 can be one of several (e.g., DICOM) images captured for a respective patient.

Image 1502 can present a baseline chest CT image showing a relatively large irregular mass in the left upper lobe (e.g., labeled with “*”) with thickening of the peribronchovascular bundle and interlobular septa. This image can represent a primary tumor with regional lymphangitic spread. Image 1504 can present another baseline chest CT image showing a dominant mass in the left lower lobe (e.g., labeled with an arrow) and multiple lung nodules.

In various implementations, the processor can feed the images 1502, 1504 as input to the model (e.g., the first model) to identify or extract one or more visual attributes or features from the images 1502, 1504. Based on the visual attributes, the processor can proceed to determine, for instance, the type of genetic mutation of the tumor.

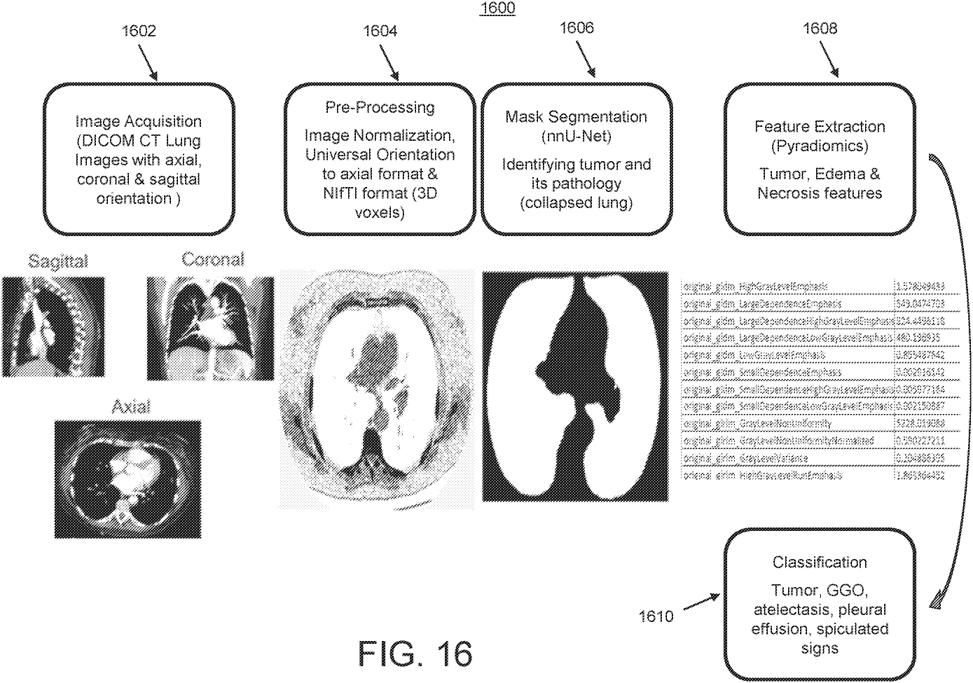

FIG. 16 illustrates an example process 1600 for image processing to extract notable features associated with certain genetic mutations mutation(s). The example process 1600 can be executed, performed, or otherwise carried out by one or more components of the system 200 (e.g., procedure identification system 202, prediction feed(s) 206, gateway service 208, etc.), the one or more components of the system 300 (e.g., analytics server 310a, system database 310b, electronic data sources 320, end-user device 340, administrator computing device 350, etc.), the computer 100, or any other computing devices described herein in conjunction with FIG. 1A-5. The example process 1600 may be performed additionally or alternatively to at least methods 400, 410, or 500, as described in conjunction with FIG. 4A-5.

At operation 1602, the processor can acquire images on the area of interest of the patient, such as the lung for a lung tumor, the brain for a brain tumor, etc. The images can include DICOM CT of the area of interest in various orientations, such as axial, coronal, and sagittal orientations. In some cases, the processor can acquire images of a single orientation for feature extraction (e.g., to output identifications of imaging proxies).

At operation 1604, the processor can pre-process the image. The processor can pre-process the image using any suitable image pre-processing technique, such as image normalization, universal orientation to axial format, and NIfTI format (e.g., 3D voxel), etc. In some cases, the processor may acquire pre-processed images from the database.

At operation 1606, the processor can perform mask segmentation on the image (e.g., pre-processed image). The processor can apply or insert the mask to filter portions of the image irrelevant to the body part of the patient (e.g., lung, brain, etc.) or the tumor at the body part of the patient. The processor can perform the mask segmentation using any suitable image masking technique. In this case, for example, the processor can perform mask segmentation to identify the tumor and its pathology.

At operation 1608, the processor can perform feature extraction using any suitable feature extraction technique, such as pyradiomic (e.g., open-source code), principle component analysis, independent component analysis, linear discriminant analysis, etc. Responsive to performing the feature extraction, the processor can generate an initial list of features extracted from the image. The features can include indications of notable irregularities in the image, such as the contrast, size, coloration, uniformity, or other variables related to the tumor.

At operation 1610, the processor can perform classification on the features using any suitable classification technique. The processor may compare the features extracted from the image to one or more reported features (e.g., known features stored in and retrieved from the database) to classify or group features relevant to certain types of genetic mutations. The reported features may refer to the historical features (e.g., measurements of features) that were stored to indicate features relevant to individual genetic mutations associated with different types of tumors. Based on the comparison, the processor can include or filter one or more features to generate a list of relevant imaging features for the type of tumor in the image.

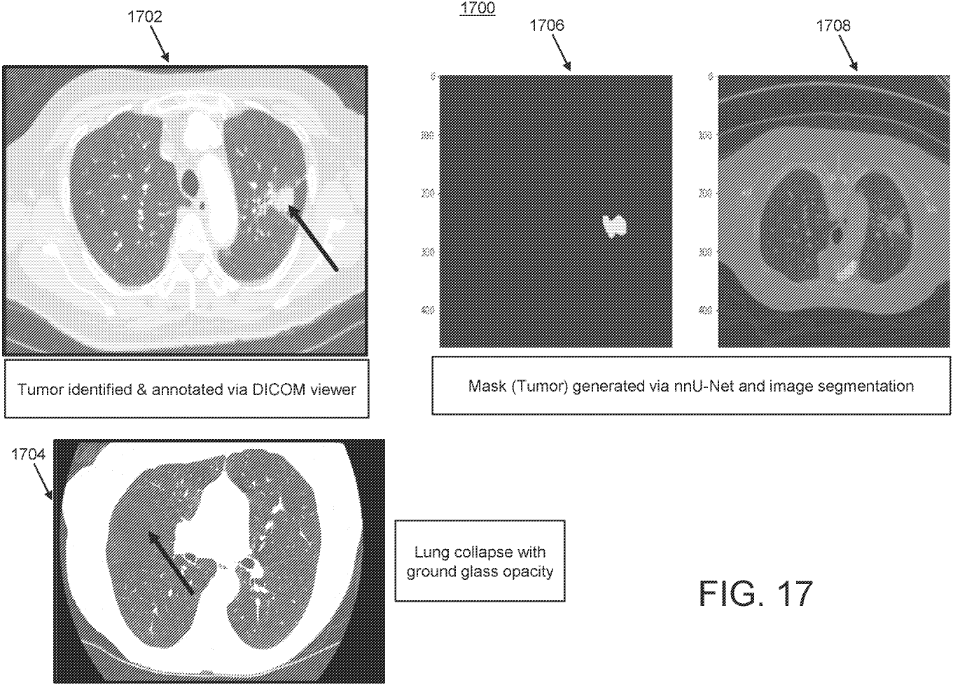

FIG. 17 illustrates an example 1700 of AI/ML generated segmented masks for validation of digital imaging and communications in medicine (DICOM) images. The example 1700 can include images 1702, 1704, 1706, and 1708. Image 1702 can include an indication of an identified tumor (e.g., labeled with an arrow) annotated via DICOM viewer. Image 1704 can include an indication of a collapsed lung with ground glass opacity (e.g., labeled with an arrow). Image 1706 can represent the result of generating and applying a mask on an image to identify the tumor via an image masking technique (e.g., nnU-net, etc.). Image 1708 can represent the result of image segmentation for the lung of the patient.

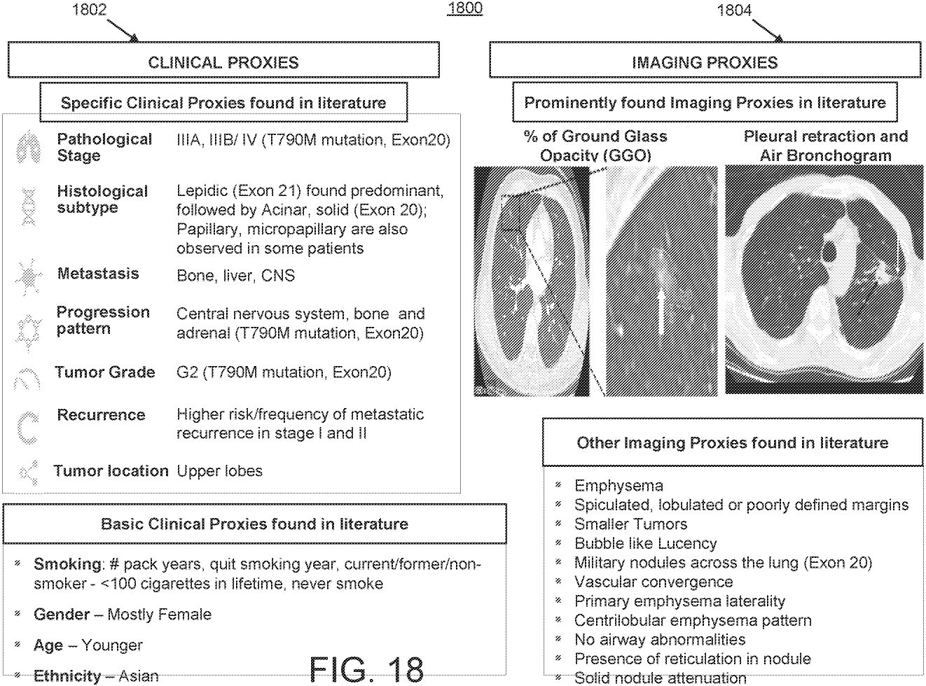

FIG. 18 illustrates examples 1800 of clinical proxies 1802 and imaging proxies 1804 for the patient. The clinical proxies 1802 can include or correspond to the result of clinical analysis performed by the HCP for the patient. The clinical proxies 1802 can include clinical information, patient attributes, or other health-related information tested or identified by the HCP. The imaging proxies 1804 can include or correspond to the output of the model, such as identifications of imaging features extracted from the images of the patient. The examples 1800 provided herein can represent a specific example for a patient with lung cancer. Other patients with different types of diseases, backgrounds, or health conditions can have different clinical proxies 1802 and imaging proxies 1804.

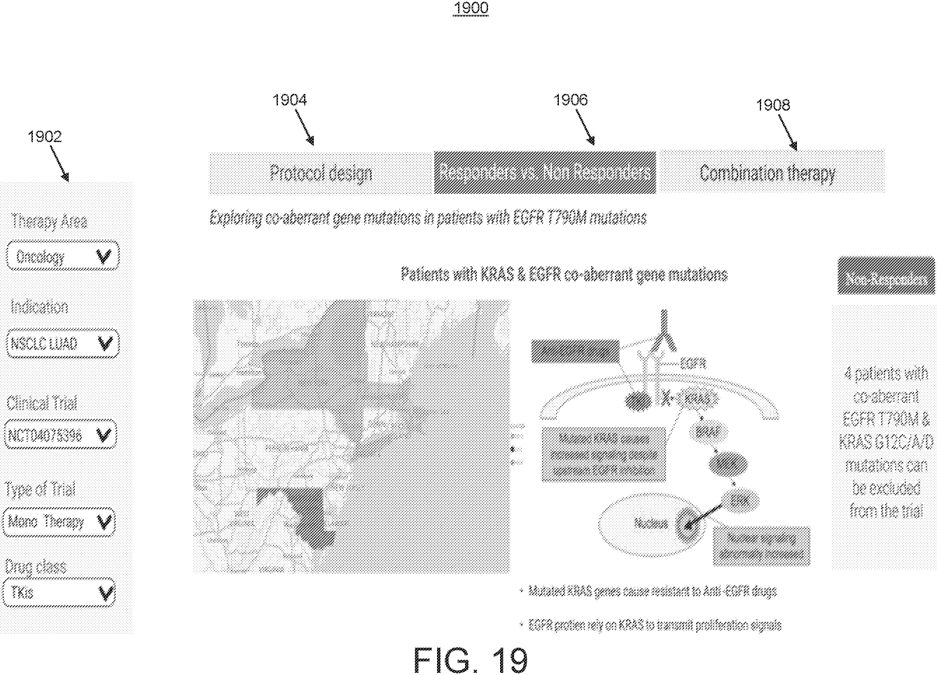

FIG. 19 illustrates an example user interface 1900 for identifying the (right) patients in a clinical trial setting. The user interface 1900 can include various interactive elements for navigating through the interfaces. The user interface 1900 can be a part of a portal for patient recruitment to a particular clinical trial based on criteria of the patient. For example, at portion 1902 of the user interface 1900, HCP can select at least one of therapy area, indication (e.g., type of tumor), clinical trial, type of trial (e.g., stage of the trial), drug class (e.g., medication), etc., to indicate the criteria for potential patients to recruit for a clinical trial. Under the category of protocol design, at portion 1904, the user interface 1900 can include information or selections related to protocol design for screening patients for the desired clinical trial. As shown in user interface 1900 of FIG. 19, after the HCP selects various criteria for the patients, the user interface 1900 can present a list of patients with a relatively high probability (e.g., confidence score) of having a particular genetic mutation to undergo a certain therapy protocol. The user interface 1900 can present the location and listing of patients that are either responders or non-responders for the clinical trial configured by the HCP, such as when the category “responders vs. non-responders” at portion 1906 is selected. The category “combination therapy” in portion 1908 can be associated with another user interface, for example, presenting different types of combination therapies available for clinical trials.

The foregoing method descriptions and the process flow diagrams are provided merely as illustrative examples and are not intended to require or imply that the steps of the various embodiments must be performed in the order presented. The steps in the foregoing embodiments may be performed in any order. Words such as “then,” “next,” etc. are not intended to limit the order of the steps; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, and the like. When a process corresponds to a function, the process termination may correspond to a return of the function to a calling function or a main function.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

References to “or” can be construed as inclusive so that any terms described using “or” can indicate any of a single, more than one, and all of the described terms. References to at least one of a conjunctive list of terms can be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

While various aspects and embodiments have been disclosed, other aspects and embodiments are contemplated. The various aspects and embodiments disclosed are for purposes of illustration and are not intended to be limiting, with the true scope and spirit being indicated by the following claims.

What we claim is:

1. A method comprising:
receiving, by a processor, an image of a tumor of a patient;
executing, by the processor, a first model to identify one or more visual attributes of the tumor using the image of the tumor as input;
executing, by the processor, a second model to predict a genetic mutation of the patient using the one or more visual attributes as input; and
identifying, by the processor, a therapy protocol associated with the tumor based on the genetic mutation.

2. The method of claim 1, the first model is a machine learning model trained based on historical data of a plurality of images of tumors, each of the plurality of images of tumors associated with a list of one or more visual attributes.

3. The method of claim 1, wherein executing the second model comprises comparing, by the processor, the one or more visual attributes to one or more templates, each template corresponding to a different type of genetic mutation.

4. The method of claim 1, wherein executing the second model comprises:
retrieving, by the processor, patient attributes of the patient from a database; and
executing, by the processor, the second model using the one or more visual attributes and the patient attributes as input.

5. The method of claim 4, wherein executing the second model comprises:
determining, by the processor, a confidence score for the genetic mutation based on the one or more visual attributes and the patient attributes; and
selecting, by the processor, the genetic mutation based on the confidence score.

6. The method of claim 1, further comprising:
adding, by the processor, an identification of the patient to a list of patients for the therapy protocol.

7. The method of claim 1, wherein the therapy protocol is a first therapy protocol, and further comprising:
executing, by the processor using one or more patient attributes of the patient as input, a third model to predict a molecular alteration for the patient; and
determining, by the processor, the first therapy protocol for the patient responsive to the molecular alteration corresponding to a second therapy protocol.

8. The method of claim 7, wherein the molecular alteration indicates a resistance to the first therapy protocol.

9. The method of claim 7, wherein the third model is a nodal data structure.

10. A system comprising:
a processor configured to:
receive an image of a tumor of a patient;
execute a first model to identify one or more visual attributes of the tumor using the image of the tumor as input;
execute a second model to predict a genetic mutation of the patient using the one or more visual attributes as input; and
identify a therapy protocol associated with the tumor based on the genetic mutation.

11. The system of claim 10, the first model is a machine learning model trained based on historical data of a plurality of images of tumors, each of the plurality of images of tumors associated with a list of one or more visual attributes.

12. The system of claim 10, wherein to execute the second model, the processor is configured to compare the one or more visual attributes to one or more templates, each template corresponding to a different type of genetic mutation.

13. The system of claim 10, wherein to execute the second model, the processor is configured to:
retrieve patient attributes of the patient from a database; and
execute the second model using the one or more visual attributes and the patient attributes as input.

14. The system of claim 13, wherein to execute the second model, the processor is configured to:
determine a confidence score for the genetic mutation based on the one or more visual attributes and the patient attributes; and
select the genetic mutation based on the confidence score.

15. The system of claim 10, wherein the processor is further configured to:
add an identification of the patient to a list of patients for the therapy protocol.

16. The system of claim 10, wherein the therapy protocol is a first therapy protocol, and the processor is further configured to:
execute, using one or more patient attributes of the patient as input, a third model to predict a molecular alteration for the patient; and
determine the first therapy protocol for the patient responsive to the molecular alteration corresponding to a second therapy protocol.

17. The system of claim 16, wherein the molecular alteration indicates a resistance to the first therapy protocol.

18. A method comprising:
receiving, by a processor, clinical information of a patient;
identifying, by the processor, one or more features from an image of a tumor of the patient;
predicting, by the processor, using a model, a genetic mutation of the patient according to the one or more features; and
determining, by the processor, a therapy protocol according to the clinical information and the genetic mutation of the patient.

19. The method of claim 18, wherein the genetic mutation is a first genetic mutation, and wherein determining the therapy protocol comprises:
predicting, by the processor, using the model, a second genetic mutation of the patient according to the one or more features; and
determining, by the processor, the therapy protocol according to a combination of the first genetic mutation and the second genetic mutation and the clinical information.

20. The method of claim 18, wherein predicting a genetic mutation and determining the therapy protocol comprises:
predicting, by the processor, using the model, more than two genetic mutations of the patient according to the one or more features; and
determining, by the processor, chemotherapy as the therapy protocol according to more than two genetic mutations associated with the tumor of the patient.
